SEQUENCE LISTING (83 sequences)

SEQ ID NO: 1 - DNA, 1677 bp, Saccharomyces cerevisiae
  source 1..1677: mol_type = genomic DNA
atggcacacatcccagaggttttacccaaatcaataccgattccggcatttattgttaccacctcatcgtacctatggtactacttcaatctggtgttgactcaaatcccgggaggccaattcatcgtttcgtacatcaagaaatcgcatcatgacgatccatacaggaccacggttgagatagggcttattttatacgggatcatctattacttgtccaagccacaacagaaaaagagtcttcaagcacagaagcccaacctatcgccccaggagattgacgcgctaattgaggactgggagcccgagcctctagtcgacccttctgccaccgatgagcaatcgtggagggtggccaaaacacccgtcaccatggaaatgcccattcagaaccatattactatcaccagaaacaacctgcaggagaagtataccaatgttttcaatttggcctcgaacaactttttgcaattgtccgctacggagcccgtgaaagaagtggtcaagaccactatcaagaattacggtgtgggcgcctgtggtcccgccgggttctacggtaaccaggacgttcattacacgttggaatatgatttagcacagttctttggcacccaaggttccgttctgtacgggcaagacttttgtgccgcaccctctgttctgcctgctttcacaaagcgtggtgatgttatcgtggcagacgaccaggtgtcattaccagtgcaaaatgctctgcaactaagcagatccacagtctactacttcaaccacaacgatatgaattcgctagaatgtttattaaacgagttgaccgaacaggagaaacttgagaaactgcccgccattccaagaaaatttatcgtcactgagggtattttccacaactcgggcgatttagctccgttgcctgagttgactaagctgaagaacaagtacaagttcagactatttgttgacgaaaccttctccattggtgttcttggcgctacgggccgtgggttgtcagagcacttcaacatggatcgcgcaactgccattgacattaccgttgggtccatggccaccgcgttggggtccaccggtggttttgtcctgggtgacagtgttatgtgtttgcaccagcgtattggttccaatgcatattgtttttctgcctgtttgccggcttacaccgtcacatccgtctccaaagtcttgaaattgatggactccaacaacgacgccgtccagacgctgcaaaaactatccaaatctttgcatgattcctttgcatctgacgactccttgcgttcatacgtaatcgtcacgtcctctccagtgtctgctgtcctacatctgcaactgactcccgcatataggtctcgcaagttcggatacacctgcgaacagctattcgaaaccatgtcagctttgcaaaagaagtcccagacaaacaaattcattgagccatacgaagaggaggaaaaatttctgcagtccatagtagatcatgctcttattaactacaacgttctcatcacaagaaacactattgttttaaaacaggagacgctaccaattgtccctagcttgaaaatctgctgtaacgccgccatgtccccagaggaactcaaaaatgcttgcgaaagtgtcaagcagtccatccttgcctgttgccaagaatctaataaataa

SEQ ID NO: 2 - protein, 558 aa, Saccharomyces cerevisiae
MAHIPEVLPKSIPIPAFIVTTSSYLWYYFNLVLTQIPGGQFIVSYIKKSHHDDPYRTTVEIGLILYGIIYYLSKPQQKKSLQAQKPNLSPQEIDALIEDWEPEPLVDPSATDEQSWRVAKTPVTMEMPIQNHITITRNNLQEKYTNVFNLASNNFLQLSATEPVKEVVKTTIKNYGVGACGPAGFYGNQDVHYTLEYDLAQFFGTQGSVLYGQDFCAAPSVLPAFTKRGDVIVADDQVSLPVQNALQLSRSTVYYFNHNDMNSLECLLNELTEQEKLEKLPAIPRKFIVTEGIFHNSGDLAPLPELTKLKNKYKFRLFVDETFSIGVLGATGRGLSEHFNMDRATAIDITVGSMATALGSTGGFVLGDSVMCLHQRIGSNAYCFSACLPAYTVTSVSKVLKLMDSNNDAVQTLQKLSKSLHDSFASDDSLRSYVIVTSSPVSAVLHLQLTPAYRSRKFGYTCEQLFETMSALQKKSQTNKFIEPYEEEEKFLQSIVDHALINYNVLITRNTIVLKQETLPIVPSLKICCNAAMSPEELKNACESVKQSILACCQESNK

SEQ ID NO: 3 - DNA, 1686 bp, Saccharomyces cerevisiae
  source 1..1686: mol_type = genomic DNA
atgagtactcctgcaaactatacccgtgtgcccctgtgcgaaccagaggagctgccagacgacatacaaaaagaaaatgaatatggtacactagattctccggggcatttgtatcaagtcaagtcacgtcatgggaagccactacctgagcccgttgtcgacacccctccttattacatttctttgttaacatatctaaattatttgattctgattatattaggtcatgttcacgacttcttaggtatgaccttccaaaaaaacaaacatctggatcttttagagcatgatgggttagcaccttggttttcaaatttcgagagtttttatgtcaggagaattaaaatgagaattgatgattgcttttctagaccaactactggtgttcctggtagatttattcgttgtattgatagaatttctcataatataaatgagtattttacctactcaggcgcagtgtatccatgcatgaacttatcatcatataactatttaggcttcgcacaaagtaagggtcaatgtaccgatgccgccttggaatctgtcgataaatattctattcaatctggtggtccaagagctcaaatcggtaccacagatttgcacattaaagcagagaaattagttgctagatttatcggtaaggaggatgccctcgttttttcgatgggttatggtacaaatgcaaacttgttcaacgctttcctcgataaaaagtgtttagttatctctgacgaattgaaccacacctctattagaacaggtgttaggctttctggtgctgctgtgcgaactttcaagcatggtgatatggtgggtttagaaaagcttatcagagaacagatagtacttggtcaaccaaaaacaaatcgtccatggaagaaaattttaatttgcgcagaagggttgttttccatggaaggtactttgtgtaacttgccaaaattggttgaattgaagaagaaatataaatgttacttgtttatcgatgaagcccattctataggcgctatgggcccaactggtcgcggtgtttgtgaaatatttggcgttgatcccaaggacgtcgacattctaatgggtactttcactaagtcgtttggtgctgctggtggttacattgctgctgatcaatggattatcgatagactgaggttggatttaaccactgtgagttatagtgagtcaatgccggctcctgttttagctcaaactatttcctcattacaaaccattagtggtgaaatatgtcccggacaaggtactgaaagattgcaacgtatagcctttaattcccgttatctacgtttagctttgcaaaggttaggatttattgtctacggtgtggctgactcaccagttattcccttactactgtattgtccctcaaagatgcccgcattttcgagaatgatgttacaaagacggattgctgttgttgttgttgcttatcctgctactccgctgatcgaatcaagagtaagattctgtatgtctgcatctttaacaaaggaagatatcgattatttactgcgtcatgttagtgaagttggtgacaaattgaatttgaaatcaaattccggcaaatccagttacgacggtaaacgtcaaagatgggacatcgaggaagttatcaggagaacacctgaagattgtaaggacgacaagtattttgttaattga

SEQ ID NO: 4 - protein, 561 aa, Saccharomyces cerevisiae
MSTPANYTRVPLCEPEELPDDIQKENEYGTLDSPGHLYQVKSRHGKPLPEPVVDTPPYYISLLTYLNYLILIILGHVHDFLGMTFQKNKHLDLLEHDGLAPWFSNFESFYVRRIKMRIDDCFSRPTTGVPGRFIRCIDRISHNINEYFTYSGAVYPCMNLSSYNYLGFAQSKGQCTDAALESVDKYSIQSGGPRAQIGTTDLHIKAEKLVARFIGKEDALVFSMGYGTNANLFNAFLDKKCLVISDELNHTSIRTGVRLSGAAVRTFKHGDMVGLEKLIREQIVLGQPKTNRPWKKILICAEGLFSMEGTLCNLPKLVELKKKYKCYLFIDEAHSIGAMGPTGRGVCEIFGVDPKDVDILMGTFTKSFGAAGGYIAADQWIIDRLRLDLTTVSYSESMPAPVLAQTISSLQTISGEICPGQGTERLQRIAFNSRYLRLALQRLGFIVYGVADSPVIPLLLYCPSKMPAFSRMMLQRRIAVVVVAYPATPLIESRVRFCMSASLTKEDIDYLLRHVSEVGDKLNLKSNSGKSSYDGKRQRWDIEEVIRRTPEDCKDDKYFVN

SEQ ID NO: 5 - DNA, 963 bp, Saccharomyces cerevisiae
  source 1..963: mol_type = genomic DNA
atgaagtttacgttagaagaccaagttgtgttgatcactggtggttcacaaggtcttggaaaggaattcgccaaaaaatattataatgaggctgaaaacacaaagattattatcgtcagtaggtcagaggctagactgctggacacatgcaacgaaattaggattgaagctcacctgagaagggaaaccactgacgagggccaagtgcaacataagttggctgcgcccttggaccttgagcaacggttattttactacccatgcgacttgtcctgctacgaatccgtggaatgtttgttcaatgccctgagagacttggatttactccctacacaaacgttatgctgtgcagggggggctgttcctaagttatttcgtgggctaagcggacatgagttgaacttgggtatggacatcaactataaaacaactttgaacgtggcacatcagattgcccttgcagagcaaaccaaggaacaccacctcatcatcttttctagtgccaccgcgctttacccatttgtgggctattcccagtatgcgcctgcaaaagctgcaatcaaatcactggtagcaatcttaagacaagaactgacgaacttccgtatcagttgtgtttatcctggtaattttgaaagcgaaggtttcactgtagagcagctaacgaaacccgaaattacaaagttgatcgaaggcccctcagacgctatcccatgcaaacaagcatgtgatatcattgccaagtcgctggccagaggtgatgatgacgtttttacagattttgtcggatggatgataatggggatggaccttgggctcaccgcaaagaaaagccgctttgttccgttgcaatggatttttggtgtcctatcaaacattctggtcgtgccattctacatggttggctgttcctggtatatcaggaaatggtttcgtgaaaatgacggcaagaaggccaactga

SEQ ID NO: 6 - protein, 320 aa, Saccharomyces cerevisiae
MKFTLEDQVVLITGGSQGLGKEFAKKYYNEAENTKIIIVSRSEARLLDTCNEIRIEAHLRRETTDEGQVQHKLAAPLDLEQRLFYYPCDLSCYESVECLFNALRDLDLLPTQTLCCAGGAVPKLFRGLSGHELNLGMDINYKTTLNVAHQIALAEQTKEHHLIIFSSATALYPFVGYSQYAPAKAAIKSLVAILRQELTNFRISCVYPGNFESEGFTVEQLTKPEITKLIEGPSDAIPCKQACDIIAKSLARGDDDVFTDFVGWMIMGMDLGLTAKKSRFVPLQWIFGVLSNILVVPFYMVGCSWYIRKWFRENDGKKAN

SEQ ID NO: 7 - DNA, 1050 bp, Saccharomyces cerevisiae
  source 1..1050: mol_type = genomic DNA
atgaacgtaacatcgaatgcaactgcagccggttcctttccactagcatttggtctcaagacctcatttgggtttatgcactatgccaaggcccctgccattaatttacgccccaaggaatccttgctgccggaaatgagtgatggtgtgctggccttggttgcgccggttgttgcctactgggcgttgtctggtatattccatgtaatagacactttccatctggctgagaagtacagaattcatccgagcgaagaggttgccaagaggaacaaggcgtcgagaatgcatgttttccttgaagtgattctacaacatatcatacagaccattgttggccttatctttatgcacttcgagccgatctacatgactgggtttgaagaaaatgccatgtggaagcttcgtgcagaccttcctcggattattccagatgccgctatttattacggctatatgtacggaatgtccgctttgaagatctttgcaggctttttattcgttgatacatggcaatactttttgcatagattgatgcatatgaataagaccttatacaaatggttccactctgttcatcatgaactatacgtgccatatgcttacggtgctcttttcaacaatcctgttgagggcttcttgttagatactttgggaaccggtattgccatgacgttaactcatttgactcacagagagcaaatcattctttttacctttgccaccatgaagactgtcgatgaccactgtgggtatgctttgccacttgacccattccaatggcttttccctaataacgctgtctatcacgatatccaccaccagcaatttggtatcaagacgaactttgctcaaccatttttcactttctgggacaatttgttccaaactaactttaaagggtttgaagaatatcaaaagaagcaaagacgtgtcaccatcgacaagtacaaagagtttttgcaagagagagaattggaaaagaaggagaaactcaaaaacttcaaagctatgaatgctgctgaaaatgaagtaaagaaagagaaataa

SEQ ID NO: 8 - protein, 349 aa, Saccharomyces cerevisiae
MNVTSNATAAGSFPLAFGLKTSFGFMHYAKAPAINLRPKESLLPEMSDGVLALVAPVVAYWALSGIFHVIDTFHLAEKYRIHPSEEVAKRNKASRMHVFLEVILQHIIQTIVGLIFMHFEPIYMTGFEENAMWKLRADLPRIIPDAAIYYGYMYGMSALKIFAGFLFVDTWQYFLHRLMHMNKTLYKWFHSVHHELYVPYAYGALFNNPVEGFLLDTLGTGIAMTLTHLTHREQIILFTFATMKTVDDHCGYALPLDPFQWLFPNNAVYHDIHHQQFGIKTNFAQPFFTFWDNLFQTNFKGFEEYQKKQRRVTIDKYKEFLQERELEKKEKLKNFKAMNAAENEVKKEK

SEQ ID NO: 9 - DNA, 978 bp, Pichia ciferrii
  source 1..978: mol_type = genomic DNA
atgtcctcccatcaattcttgatcaatcaaactactttggctgctccaccagttcatttggttgaaaaaccatctttgatcaacggtatcccagataacattttggctttgattgctccagttatcgcctactattcttactctggtttcttctacgttatcgacaccttggaaattgccgaattatacagaattcacccaccagaagaagtctccagtagaaacaaagctactaagttcgatgttttgaaggacgttgtcttgcaacacttcatccaatctgttgttggttacatcttcacctacttcgacccaattcaatacactggtgatgaagaatatcaagcctggaagttgcaacaaactttgccatttttgcctttcgatgttgcttactactggaatatgtatggttggtcctgtttgaagattggtttggccttcttgattatcgactcttggcaatattggttgcacagaatcatgcatttgaacaagaccttgtacaaaagattccactccagacaccacagattatatgttccatatgcttttggtgccttgtataacgatccattcgaaggttttttgttggatactttgggtactggtattgctgctatcgttactcaattgactccaagagaatccattgtcttgtacactttctctaccttgaaaaccgttgatgatcattgcggttattccttgccatatgatccattccaaatcttgttcccaaacaactccatctaccatgatatccatcatcaacaattcggtatcaagaccaacttctctcaaccatttttcacccattgggacgttttctctaacaccagatacaaagaaatcgacgaatacagagaaaagcaaaaggctattaccattgccaagtacaaagaatttttacacgacagagaaatcgccaagcaaaagaagaaagctgaaatctacaaggacaaaaagactgattaa

SEQ ID NO: 10 - protein, 325 aa, Pichia ciferrii
MSSHQFLINQTTLAAPPVHLVEKPSLINGIPDNILALIAPVIAYYSYSGFFYVIDTLEIAELYRIHPPEEVSSRNKATKFDVLKDVVLQHFIQSVVGYIFTYFDPIQYTGDEEYQAWKLQQTLPFLPFDVAYYWNMYGWSCLKIGLAFLIIDSWQYWLHRIMHLNKTLYKRFHSRHHRLYVPYAFGALYNDPFEGFLLDTLGTGIAAIVTQLTPRESIVLYTFSTLKTVDDHCGYSLPYDPFQILFPNNSIYHDIHHQQFGIKTNFSQPFFTHWDVFSNTRYKEIDEYREKQKAITIAKYKEFLHDREIAKQKKKAEIYKDKKTD

SEQ ID NO: 11 - DNA, 1236 bp, Saccharomyces cerevisiae
  source 1..1236: mol_type = genomic DNA
atgacatcagctacggacaaatctatcgataggttagttgttaatgcaaaaacaagaagacgaaactcttccgtgggtaaaattgatttaggtgatacagttcctggctttgcagccatgcctgaaagtgctgcctctaaaaatgaggccaaaaaaaggatgaaagccttgactggtgactctaaaaaggatagtgacctactgtggaaggtttggttttcatatagagaaatgaattaccgtcatagttggttgacaccattcttcatacttgtatgcgtgtatagcgcgtactttttatctgggaatagaacagaatcaaacccgctgcacatgttcgtagccatatcatatcaagttgatggcacagactcatatgcaaaaggtatcaaagatttgagttttgtgtttttctacatgattttcttcacatttttacgtgagtttttgatggatgttgtaattcgaccattcacggtatacctaaatgttacttccgagcatcgtcaaaagcgtatgctagaacaaatgtatgccatattttattgcggagtttcagggccctttggtctttatattatgtaccatagtgatttgtggttgttcaagacaaaaccaatgtacagaacatatcctgttataaccaatccgttcttgtttaagatattttacttgggtcaagcggcattttgggcgcaacaggcttgtgttcttgttctacaattagaaaagccaagaaaggattacaaggaattggtttttcatcacattgtgacattattattaatttggtcatcatatgttttccattttaccaaaatgggattggctatctatattactatggatgtgtcagattttttcctttctttgtctaagacattaaactatctgaattctgtatttactccctttgtgttcggcttgttcgtgttcttttggatctatctgcgccatgtcgtgaatatcagaatattatggtcagtcttaacagaattccgtcatgaaggtaattatgtgttgaattttgccacacaacaatacaaatgttggatttcgttgccaattgtatttgtactaattgctgcgttacaattagttaacctgtattggctgtttttaattcttagaatcttgtacagattgatatggcaaggtatccaaaaggacgaaagaagtgacagtgattctgatgagagcgctgaaaatgaagaatctaaggaaaagtgtgaataa

SEQ ID NO: 12 - protein, 411 aa, Saccharomyces cerevisiae
MTSATDKSIDRLVVNAKTRRRNSSVGKIDLGDTVPGFAAMPESAASKNEAKKRMKALTGDSKKDSDLLWKVWFSYREMNYRHSWLTPFFILVCVYSAYFLSGNRTESNPLHMFVAISYQVDGTDSYAKGIKDLSFVFFYMIFFTFLREFLMDVVIRPFTVYLNVTSEHRQKRMLEQMYAIFYCGVSGPFGLYIMYHSDLWLFKTKPMYRTYPVITNPFLFKIFYLGQAAFWAQQACVLVLQLEKPRKDYKELVFHHIVTLLLIWSSYVFHFTKMGLAIYITMDVSDFFLSLSKTLNYLNSVFTPFVFGLFVFFWIYLRHVVNIRILWSVLTEFRHEGNYVLNFATQQYKCWISLPIVFVLIAALQLVNLYWLFLILRILYRLIWQGIQKDERSDSDSDESAENEESKEKCE

SEQ ID NO: 13 - DNA, 1257 bp, Saccharomyces cerevisiae
  source 1..1257: mol_type = genomic DNA
atgtcgacaataaagccaagcccttcaaacaacaatttgaaagtgaggtcaagaccaagacgcaagtcttccataggtaagatcgacctcggggacactgtacctagtttaggtactatgttcgagacaaaagaatctaaaacggctgctaaaaggcgtatgcagagattatctgaagccaccaaaaatgatagcgacctggtcaagaaaatctggttttcattccgagagataagttaccgtcatgcctggatagctcccttgatgattttaatcgcggtatatagtgcctatttcacttctggtaacactacaaagacaaatgtgttacatagatttgtcgctgtgtcataccaaataggtgataccaacgcatatggtaaaggtattaacgatttgtgctttgtattttactatatgatttttttcacattcttacgtgaattcctaatggatgttgtcattagaccgttcgctattaggttacacgtcacttctaagcacagaataaaaagaatcatggaacaaatgtacgctattttctacaccggtgtttccggtcctttcgggatatattgtatgtaccattcggatttatggtttttcaacacaaaggcaatgtacagaacatatccagatttcactaatcccttcttattcaaggtattctatttgggtcaagctgcattttgggctcaacaagcctgcattcttgtattgcaactagaaaagccaagaaaagatcataacgagttgactttccatcatattgttactttactattgatttggtcttcctacgtatttcatttcactaaaatgggactgccaatctatatcacaatggatgtttctgattttcttttgtcattctccaaaactctaaattatttggactctggattggcttttttttcttttgctatctttgttgttgcgtggatttatttgcgtcattacataaatttaaagatcctatggtccgttttaacgcaatttcgcacggaaggtaactatgtcttgaattttgccactcagcaatataaatgttggatttccttaccaattgtctttgtattgataggcgctttgcaattagtgaatctttattggctgttccttatttttagagttctttacaggattctatggagaggtattctcaaggatgacagaagtgatagtgaatcagacgaagaaagtgatgaaagttccactactccaactgacagtactccaacgaaaaaggatatttga

SEQ ID NO: 14 - protein, 418 aa, Saccharomyces cerevisiae
MSTIKPSPSNNNLKVRSRPRRKSSIGKIDLGDTVPSLGTMFETKESKTAAKRRMQRLSEATKNDSDLVKKIWFSFREISYRHAWIAPLMILIAVYSAYFTSGNTTKTNVLHRFVAVSYQIGDTNAYGKGINDLCFVFYYMIFFTFLREFLMDVVIRPFAIRLHVTSKHRIKRIMEQMYAIFYTGVSGPFGIYCMYHSDLWFFNTKAMYRTYPDFTNPFLFKVFYLGQAAFWAQQACILVLQLEKPRKDHNELTFHHIVTLLLIWSSYVFHFTKMGLPIYITMDVSDFLLSFSKTLNYLDSGLAFFSFAIFVVAWIYLRHYINLKILWSVLTQFRTEGNYVLNFATQQYKCWISLPIVFVLIGALQLVNLYWLFLIFRVLYRILWRGILKDDRSDSESDEESDESSTTPTDSTPTKKDI

SEQ ID NO: 15 - DNA, 453 bp, Saccharomyces cerevisiae
  source 1..453: mol_type = genomic DNA
atgtctcaacccactcccatcataactacaaaatcagctgctaagccaaaaccaaaaatttttaatttattccgcgtttgcttcatttcattattgctgatcgctgcggttgaatacttcaagtatggtacaagaattaactatgaatggttccattgtaccccaatcaaagaaccccagtctggctcagtaatcaagctttgggcacgtggtgggccaagttgtgacaaaagaggcgaatataaaactatagtaaagagaatcactagagattatgaaccaaatgatgaacatctctcgttctgtatcatcgagaatgataatgttccacccgtccactacccaattcacgaagataaaggtgaacctggctacgtagcttatgtcgggtacgacacagactctgagctggttcaagaactatgtgctgattccacaatttatcacatgtga

SEQ ID NO: 16 - protein, 150 aa, Saccharomyces cerevisiae
MSQPTPIITTKSAAKPKPKIFNLFRVCFISLLLIAAVEYFKYGTRINYEWFHCTPIKEPQSGSVIKLWARGGPSCDKRGEYKTIVKRITRDYEPNDEHLSFCIIENDNVPPVHYPIHEDKGEPGYVAYVGYDTDSELVQELCADSTIYHM

SEQ ID NO: 17 - DNA, 1188 bp, Saccharomyces cerevisiae
  source 1..1188: mol_type = genomic DNA
atgtctttggaaagagaggaaccacaacatttcggagcagggccagctcaaatgcctacaccagttttgcaacaagctgctaaagacttaatcaatttcaatgacataggtttgggtatcggtgaaatttctcaccgttcgaaggatgccaccaaagtgattgaagactctaagaagcacttaatcgaactgctaaatattcctgacactcatgaagtgttctacttgcaaggtggtggcactactggtttttcttccgttgctactaatttggcagctgcatatgtgggtaagcatgggaagattgcacctgccggttatttagtcaccggtagttggtctcagaaatcttttgaagaggcaaagagattgcacgttcctgctgaagttatcttcaacgctaaagattataacaatggcaaatttggtaaaattccggatgaatccctttgggaagataaaatcaaaggtaaggctttctcatacgtgtacctatgtgagaatgaaactgttcatggtgttgaatggccagaattaccaaaatgtttagtaaacgaccccaacatcgaaattgttgctgacttatccagcgacattttgtctcgtaagattgacgtttctcaatacggtgtcatcatggcaggcgcccaaaaaaacattggtttagcaggcttaaccctatacattatcaagaaatccatccttaagaatatttctggcgcttctgatgaaacattacatgaattgggagtaccaatcacccctattgcattcgactatccaacggtggtgaagaacaactcagcctataatacaattccaattttcactttacatgttatggatctcgtgttccaacatattttgaagaagggtggtgttgaagcgcaacaggctgaaaatgaagaaaaggccaagatattatatgaggcattggatgcaaattcagatttttacaacgttccagtggatccaaagtgtagatcaaaaatgaatgtcgttttcacccttaaaaaggacggccttgatgaccagtttctaaaagaggcagctgctcgtcatttaaccggtttgaaaggacatcgttcagttggtgggttcagagcctccatctataacgcgctttcagtgaaagctgtacaaaacttggtagattttatcaaggaatttgctgagaaaaacgcttaa

SEQ ID NO: 18 - protein, 395 aa, Saccharomyces cerevisiae
MSLEREEPQHFGAGPAQMPTPVLQQAAKDLINFNDIGLGIGEISHRSKDATKVIEDSKKHLIELLNIPDTHEVFYLQGGGTTGFSSVATNLAAAYVGKHGKIAPAGYLVTGSWSQKSFEEAKRLHVPAEVIFNAKDYNNGKFGKIPDESLWEDKIKGKAFSYVYLCENETVHGVEWPELPKCLVNDPNIEIVADLSSDILSRKIDVSQYGVIMAGAQKNIGLAGLTLYIIKKSILKNISGASDETLHELGVPITPIAFDYPTVVKNNSAYNTIPIFTLHVMDLVFQHILKKGGVEAQQAENEEKAKILYEALDANSDFYNVPVDPKCRSKMNVVFTLKKDGLDDQFLKEAAARHLTGLKGHRSVGGFRASIYNALSVKAVQNLVDFIKEFAEKNA

SEQ ID NO: 19 - DNA, 930 bp, Saccharomyces cerevisiae
  source 1..930: mol_type = genomic DNA
atgtcaaagtttgttatcacctgcatagctcatggagaaaatctcccaaaagaaaccatcgaccagattgcgaaagaaattactgaaagctcggcgaaagatgtatcaatcaatggtaccaagaaactatcggccagagctaccgatatattcattgaagtcgcaggatcgattgttcaaaaagacctcaagaataagctgacgaacgtcattgacagtcataatgatgttgatgttattgtttctgtcgacaatgaatatcgtcaagccaaaaagctctttgtctttgacatggattcaacattaatctaccaagaggtcatcgaattgattgccgcttatgctggtgttgaagaacaagtgcacgagatcacagaaagagccatgaacaatgaacttgatttcaaagagtctttaagagaacgcgttaaattattgcaaggcctccaagtcgatacactatatgatgaaataaaacaaaagctagaggtcaccaagggtgtgccagaactatgcaagttccttcacaaaaaaaattgcaagctcgctgttttaagcggtggttttattcagtttgccggttttatcaaggatcagttaggtttagatttttgtaaggcaaacttgttggaagttgacactgatggaaaattaaccggtaaaacactgggtcctatcgtagacggacagtgcaagagtgaaacccttctgcaactatgtaatgactacaatgttccagttgaagcaagttgtatggtgggtgacggtggtaacgacctgccagccatggctaccgccgggtttgggatcgcatggaacgccaagccaaaggtgcagaaagctgcaccttgtaagttgaataccaagagcatgactgacattttatacattcttggttacaccgatgatgaaatatacaatagacaatga

SEQ ID NO: 20 - protein, 309 aa, Saccharomyces cerevisiae
MSKFVITCIAHGENLPKETIDQIAKEITESSAKDVSINGTKKLSARATDIFIEVAGSIVQKDLKNKLTNVIDSHNDVDVIVSVDNEYRQAKKLFVFDMDSTLIYQEVIELIAAYAGVEEQVHEITERAMNNELDFKESLRERVKLLQGLQVDTLYDEIKQKLEVTKGVPELCKFLHKKNCKLAVLSGGFIQFAGFIKDQLGLDFCKANLLEVDTDGKLTGKTLGPIVDGQCKSETLLQLCNDYNVPVEASCMVGDGGNDLPAMATAGFGIAWNAKPKVQKAAPCKLNTKSMTDILYILGYTDDEIYNRQ

SEQ ID NO: 21 - DNA, 1410 bp, Saccharomyces cerevisiae
  source 1..1410: mol_type = genomic DNA
atgacaagcattgacattaacaacttacaaaatacctttcaacaagctatgaatatgagcggctccccaggcgctgtttgtacttcacctacgcaatctttcatgaataccgttccacagcgcttgaatgctgtaaagcacccaaaaattttgaagcctttctcaacgggtgatatgaagattttactattagaaaacgttaatcaaactgctattacaatcttcgaagagcaaggttaccaagtcgaattctataaatcttcattgcccgaggaagagttgatcgaaaagatcaaggacgttcatgctattggtatcagatcaaagactagattaacttcaaatgtcttacaacatgcgaagaatctggtttgtattggttgtttctgtatcggtaccaaccaagttgacttagactacgctaccagcagaggtattgctgttttcaactcgcctttctccaactcaagatcagtagcagaattggtcatcgctgaaatcattagtttagcaagacaactaggtgatagatctatcgaattacataccggtacatggaataaggttgctgctagatgttgggaggtaagaggaaaaactcttggtattattgggtacggtcacattggttcccaattatcagttcttgcagaagctatgggtttgcatgtgttgtactacgatattgtaactatcatggccttgggtactgccagacaagtttctacattagatgaattgttgaataaatctgattttgtgacactacatgtaccagctactcctgaaactgaaaaaatgttatctgccccacaatttgctgctatgaaggatggcgcttatgttattaatgcttcaagaggtactgtcgtggacattccatctttgatccaagccgtgaaagccaacaaaattgcaggtgctgctttggatgtttatccacatgaaccagctaagaacggtgaaggttcatttaacgatgagctaaatagctggacttctgaattagtttcattaccaaatatcatcttgacaccacacattggtggctctaccgaagaagcccaaagctcaatcggtattgaagtggctaccgcattgtccaaatacatcaatgaaggtaactctgtcggttcagtcaacttcccagaagtggcattgaaatcattgtcttacgaccaagagaacactgtgcgtgtgttatacattcaccaaaatgtaccaggtgttttgaagaccgtcaatgatattttatcgaaccataacatcgaaaagcaattttccgattcaaatggtgaaattgcttatttaatggctgatatctcttctgttgaccaaagcgatattaaagatatttatgaacaactaaatcaaacctctgctaagatctcaattagattgctatattaa

SEQ ID NO: 22 - protein, 469 aa, Saccharomyces cerevisiae
MTSIDINNLQNTFQQAMNMSGSPGAVCTSPTQSFMNTVPQRLNAVKHPKILKPFSTGDMKILLLENVNQTAITIFEEQGYQVEFYKSSLPEEELIEKIKDVHAIGIRSKTRLTSNVLQHAKNLVCIGCFCIGTNQVDLDYATSRGIAVFNSPFSNSRSVAELVIAEIISLARQLGDRSIELHTGTWNKVAARCWEVRGKTLGIIGYGHIGSQLSVLAEAMGLHVLYYDIVTIMALGTARQVSTLDELLNKSDFVTLHVPATPETEKMLSAPQFAAMKDGAYVINASRGTVVDIPSLIQAVKANKIAGAALDVYPHEPAKNGEGSFNDELNSWTSELVSLPNIILTPHIGGSTEEAQSSIGIEVATALSKYINEGNSVGSVNFPEVALKSLSYDQENTVRVLYIHQNVPGVLKTVNDILSNHNIEKQFSDSNGEIAYLMADISSVDQSDIKDIYEQLNQTSAKISIRLLY

SEQ ID NO: 23 - DNA, 951 bp, Saccharomyces cerevisiae
  source 1..951: mol_type = genomic DNA
atgggaatatttcgttggaactatccagagagttctgtccccggcgtttggggagaaacaacttccactattgactggtgtgaggagaactatgtcgtttctccctatattgccgagtggtcaaacactttaactaacagcgtattcatactgtcagcgatttacacaacttactctgcttacaagaataaattagaaaaaaggtttttacttattggcttcgggtacggtttggtcggagtaggatcatggctatttcatatgacactgaagtatagattccaactattggatgaacttccaatgatatacgccatgtgcattccgacatggagtttagtatgcgaggccaaagaggcattacttaacggagataatcacaagaaggttcctctatttgaacagatattcatcggcgtaattatcggcctggccgttacaacagcaagcatactctacgttatttacaaaaatgtcgatatccatcaaattttgtttggcgtacagattgtagttgtggctgctactgcaggaagtttgacgtacagatacgtccatgatccacttgccaaaagaaatctcaaggcttcaatggcgctcggcgcaattttgttcttatctggctacatttcgtggctacttgatatacactattgttcgttctgggtgcacgttagaagaagtattttggctttaccacttggtgtactgcttgaaccacacggatggtggcatatattaactggtatggggatttatttctacattgtttctttggaacatttaagggtcattacgctcaacgtcagctgcaattaccagttcatctggagatggaaagtcttccctgaactgatatggaaagggcgcaaaccctcaacaagatattcacttgaactatttggcccatacgtagaagatcaatcaattgaagttaaaaaggagaagtaa

SEQ ID NO: 24 - protein, 316 aa, Saccharomyces cerevisiae
MGIFRWNYPESSVPGVWGETTSTIDWCEENYVVSPYIAEWSNTLTNSVFILSAIYTTYSAYKNKLEKRFLLIGFGYGLVGVGSWLFHMTLKYRFQLLDELPMIYAMCIPTWSLVCEAKEALLNGDNHKKVPLFEQIFIGVIIGLAVTTASILYVIYKNVDIHQILFGVQIVVVAATAGSLTYRYVHDPLAKRNLKASMALGAILFLSGYISWLLDIHYCSFWVHVRRSILALPLGVLLEPHGWWHILTGMGIYFYIVSLEHLRVITLNVSCNYQFIWRWKVFPELIWKGRKPSTRYSLELFGPYVEDQSIEVKKEK

SEQ ID NO: 25 - DNA, 1341 bp, Saccharomyces cerevisiae
  source 1..1341: mol_type = genomic DNA
atgaatgccctaaaatatttctcaaatcatttaataactacaaagaaacaaaaaaaaatcaatgttgaggtgacaaagaatcaagatctactaggcccttcaaaggaagtttcgaacaaatatactagccacagcgaaaatgattgcgttagtgaggtggaccaacagtacgaccactcctcaagccacttaaaagaatctgatcaaaatcaagagcgcaaaaattctgttcctaaaaaaccaaaagctttacgttccatcctaatagagaaaatagcgtcaattttgtgggcactactactttttcttccgtattatctgataatcaagcccttgatgtctttatggtttgtttttaccttcccactaagtgtcatcgagcgtcgtgtaaaacacaccgataagagaaacaggggttcaaatgctagtgagaatgagctgcctgtcagttcaagcaatattaacgattctagcgagaaaacaaatcctaaaaattgcaatctaaatacgattcccgaggcagttgaggatgacttaaatgccagtgatgagataattctacaaagggataatgttaaaggctcattacttagagctcaatctgtcaaatcaaggccaagaagttattccaaatcagaactatcactttctaatcattcaagttctaataccgtatttggtacaaaacgaatgggaaggttcttatttccaaagaagctgatacctaaatccgtgcttaacacacaaaagaaaaagaaactggttatagatctcgatgagaccttaattcactcggcttctcgaagtacaacgcatagtaattcttctcagggtcacttggtggaagtgaagtttggattgagcggaattcgtacactatactttattcacaagaggccttactgtgatttatttttgacaaaagttagcaaatggtacgaccttatcatttttacagcatccatgaaagaatacgctgatcctgtgatagattggttagaaagctccttcccatccagtttttcaaaaagatattaccgttctgattgcgttctaagggatggtgttggatacattaaagatttgagcattgtcaaggactccgaagaaaacgggaaaggcagctcctcttctttggatgatgtcattattatagataacagcccggtaagttacgcaatgaatgtagataacgctattcaagtagaaggatggataagtgatccaactgatacggacttactgaacctgttacctttcttggaggctatgaggtattcaacagatgtcaggaatatactggcattgaaacatggagagaaggcattcaacataaactga

SEQ ID NO: 26 - protein, 446 aa, Saccharomyces cerevisiae
MNALKYFSNHLITTKKQKKINVEVTKNQDLLGPSKEVSNKYTSHSENDCVSEVDQQYDHSSSHLKESDQNQERKNSVPKKPKALRSILIEKIASILWALLLFLPYYLIIKPLMSLWFVFTFPLSVIERRVKHTDKRNRGSNASENELPVSSSNINDSSEKTNPKNCNLNTIPEAVEDDLNASDEIILQRDNVKGSLLRAQSVKSRPRSYSKSELSLSNHSSSNTVFGTKRMGRFLFPKKLIPKSVLNTQKKKKLVIDLDETLIHSASRSTTHSNSSQGHLVEVKFGLSGIRTLYFIHKRPYCDLFLTKVSKWYDLIIFTASMKEYADPVIDWLESSFPSSFSKRYYRSDCVLRDGVGYIKDLSIVKDSEENGKGSSSSLDDVIIIDNSPVSYAMNVDNAIQVEGWISDPTDTDLLNLLPFLEAMRYSTDVRNILALKHGEKAFNIN

SEQ ID NO: 27 - DNA, 780 bp, Saccharomyces cerevisiae
  source 1..780: mol_type = genomic DNA
atggagccagagagcataggcgatgtggggaaccatgcccaggatgatagtgccagtatagtgtccgggcctcgcaggcgttctactagcaagacatccagtgcgaagaatatacggaactccagtaatatctctccagcatcgatgattttcaggaatttgttgatactggaggatgatttaagacgccaagctcacgaacaaaagatactgaagtggcaattcactttgttcttagcgtctatggccggtgtaggcgcatttaccttctacgaactttatttcacttcagattatgtcaagggcctccatagggttattttgcaattcactctttctttcatttccattactgtagttctttttcatatcagtggacaatatagaagaactatcgtcattccaagaagattttttacctctactaataaagggattaggcagtttaatgtgaagctagttaaagtacagtctacgtgggacgagaaatacacagattcagtaagatttgtgagtcgaacaattgcttattgtaatatttattgtttgaaaaaatttctgtggcttaaagacgataatgccattgtgaaattttggaaaagtgtcacgatacaatcccaaccgaggatcggagctgtggatgtgaaattagtcctcaaccccagagcatttagtgcagagattagagaaggatgggagatttatagagacgagttttgggccagggaaggtgctagaagacgcaaacaagcgcacgaactccgacctaaatcagaatga

SEQ ID NO: 28 - protein, 259 aa, Saccharomyces cerevisiae
MEPESIGDVGNHAQDDSASIVSGPRRRSTSKTSSAKNIRNSSNISPASMIFRNLLILEDDLRRQAHEQKILKWQFTLFLASMAGVGAFTFYELYFTSDYVKGLHRVILQFTLSFISITVVLFHISGQYRRTIVIPRRFFTSTNKGIRQFNVKLVKVQSTWDEKYTDSVRFVSRTIAYCNIYCLKKFLWLKDDNAIVKFWKSVTIQSQPRIGAVDVKLVLNPRAFSAEIREGWEIYRDEFWAREGARRRKQAHELRPKSE

SEQ ID NO: 29 - DNA, 1875 bp, Saccharomyces cerevisiae
  source 1..1875: mol_type = genomic DNA
atggtggtgcagaaaaaacttagggctatcttgaccgatgaaggtgtattgatcaaatcgcaatcacaccatatgttcaataagcatggtcaactcagaagcggagattctttatccttgttgagctgcttgtcctgtctggatgatggaactttgagctctgatggaggttcttttgatgaggatgattccctggaactgttgcctcttaatactaccattccgttcaacagaattttgaacgcaaaatatgtgaatgtcggtcagaaaggcttcaataatggcaaaatttcttcgaatccttttcaaacggaaaatctgagttcttcgtctgaaaatgacgacgttgagaatcatagtttgagcaatgataaggctcctgtaagcgaatcacagtcatttcccaaaaaagacaagtgggatacaaaaacgaacactgtgaaggtgtctcccgatgattcacaggataactcaccatctttagggataaaagataatcaacagttaattgagttaacttttgctgtacccaagggccatgatgttataccacaaaaattaaccttgttaatagatcacgtttctaggaaatcgagagcaaataccggagaggagaacatttcttctggtactgtggaagaaatcctggaaaaaagttatgaaaattccaagagaaacagatcgatattagtcattattaatccccacggtggtaaaggtactgctaaaaatttattcctgacaaaagcaaggccaatactagtggaaagtggctgcaaaatagaaattgcatacacaaaatatgcccgtcacgccatcgatattgccaaagatttagatatcagcaaatacgataccattgcatgtgcctcgggtgatggtattccatacgaagtaattaatgggctttatagaagacccgacagagtggatgcgttcaataaactagccgtaactcagctaccttgcggttcaggaaatgctatgagcatttcatgtcattggacaaataacccatcgtacgccgctctgtgccttgtcaaatccattgaaacaagaatagacttaatgtgttgttcccaaccttcttatatgaacgaatggccaagattatcctttttgagtcagacgtacggcgttattgcagaatctgatattaacactgaattcatcagatggatgggtcccgttaggtttaatttgggtgtagcattcaacattatccaaggtaagaaatatccctgcgaagttttcgtcaaatatgctgccaaatctaaaaaggaattaaaagttcatttcttagaaaataaagataaaaacaaaggatgtttaaccttcgaaccaaatcctagcccaaactcttcgccggatttactatctaaaaacaatatcaacaacagtacaaaagatgaactttcaccgaattttctcaacgaggacaactttaaattaaagtatccgatgacggaaccagtacctagagactgggagaaaatggattcagagctaactgataacttaacaatcttttacacagggaaaatgccgtatattgctaaggacaccaaattttttcccgctgctttaccagcggatggtaccattgatttagtcataacggatgcaagaatcccagtgacaagaatgacaccaattttattatccttggataaaggttctcatgtattagagccagaagttattcactcaaaaatattggcttataagattataccaaaagtggagtcaggtttattttcagtggatggtgaaaagtttcctttggaacccttgcaagtggaaataatgcccatgttatgcaagacgttgctaaggaatggtagatatatcgatacagagtttgaatccatgtag

SEQ ID NO: 30 - protein, 624 aa, Saccharomyces cerevisiae
MVVQKKLRAILTDEGVLIKSQSHHMFNKHGQLRSGDSLSLLSCLSCLDDGTLSSDGGSFDEDDSLELLPLNTTIPFNRILNAKYVNVGQKGFNNGKISSNPFQTENLSSSSENDDVENHSLSNDKAPVSESQSFPKKDKWDTKTNTVKVSPDDSQDNSPSLGIKDNQQLIELTFAVPKGHDVIPQKLTLLIDHVSRKSRANTGEENISSGTVEEILEKSYENSKRNRSILVIINPHGGKGTAKNLFLTKARPILVESGCKIEIAYTKYARHAIDIAKDLDISKYDTIACASGDGIPYEVINGLYRRPDRVDAFNKLAVTQLPCGSGNAMSISCHWTNNPSYAALCLVKSIETRIDLMCCSQPSYMNEWPRLSFLSQTYGVIAESDINTEFIRWMGPVRFNLGVAFNIIQGKKYPCEVFVKYAAKSKKELKVHFLENKDKNKGCLTFEPNPSPNSSPDLLSKNNINNSTKDELSPNFLNEDNFKLKYPMTEPVPRDWEKMDSELTDNLTIFYTGKMPYIAKDTKFFPAALPADGTIDLVITDARIPVTRMTPILLSLDKGSHVLEPEVIHSKILAYKIIPKVESGLFSVDGEKFPLEPLQVEIMPMLCKTLLRNGRYIDTEFESM

SEQ ID NO: 31 - DNA, 2064 bp, Saccharomyces cerevisiae
  source 1..2064: mol_type = genomic DNA
atgactttgaaaccttcaaagagacgtaagggcaggtctcgccattccaggaagaagcaaataacgtcagcgatactgactgaggaaggaataatgatcaaggcaaaaccatcaagtccttacacatacgcaaatagaatggcagataaacgaagtcgcagcagcattgacaacatcagtagaactagctttcaaagcaacatcagtagaactagctttcaaagcaacagtgataacaacagtatatttgaaacggcttcactaattagctgtgttacctgtttaagcgatactgatacaatagacagatcggaaacatcgacaacggatacaagtaaagatgatctttctgctaatccaaaacttcattatccttcggtgaatggtcaattgccagcaaacaccgttatcccctatggacgaattctggatgccagatacattgaaaaggaacctctgcattattatgatgccaattcatcacccagttcacctttaagcagctcaatgagtaacattagtgaaaagtgtgatcttgatgaattagagtcttcccaaaaaaaagaaaggaagggcaactcgctatcgcgaggaagtaacagtagtagtagcctcctgacttccagatctccttttacgaaactagtagaggttatatttgctaggccaagacggcatgacgttgtacccaaaagggtttcattgtatattgactataaaccccattcatcttctcacttaaaagaagaagatgacttggttgaggagattttaaagagaagctacaaaaacactagaaggaacaaatccatatttgtgatcattaatccgtttggtggtaaaggtaaggcgaaaaaactgtttatgacaaaggcaaagccgttactattagcaagtcggtgttccatagaagtggtttatacaaaataccctggtcatgctatagagatcgcgcgggaaatggatattgacaaatatgacactattgcttgcgcttcgggagatggcattcctcatgaggtgatcaatgggttataccaaaggcctgatcatgtcaaagcattcaacaatatcgccattacagaaattccatgcggatcaggtaacgcaatgagcgtatcctgccactggacaaacaatccttcgtactcaactttatgcttaattaaatcgatagagactagaattgatttgatgtgttgttcgcagccttcttatgcaagagagcacccaaagttatcatttttaagtcaaacatatggtctcattgcagaaactgacataaacactgaatttattagatggatgggacctgcaaggtttgaattgggtgtagccttcaatatcatacaaaaaaaaaaatatccttgtgagatatatgtaaagtatgctgccaaatcaaaaaacgagttaaaaaatcattacctggaacacaaaaataaagggtcgttagaattccagcatattactatgaacaaagataacgaggattgtgataattacaattacgaaaatgaatacgaaaccgaaaacgaagatgaagatgaagatgcggatgcggatgacgaagactcccacttgatatctcgtgatctggcagattctagtgctgatcaaattaaagaggaagatttcaaaataaaatatccattagatgaaggtatccctagtgactgggaaagattggatcctaatatttcgaacaacctaggtatcttctatacgggtaaaatgccatatgtggctgctgacactaaattctttccggcagcgcttccttcagatggtacaatggatatggttatcaccgatgcaagaacctcgttgacgaggatggcaccaatcctgctgggactagataagggttcccatgttttacaaccggaagtcttacactctaaaattttggcatacaagataataccaaagctagggaacggcttgttctctgtcgatggcgagaaatttcctctagagccccttcaagtcgaaattatgccacgcttatgcaagacgttactgagaaatggccgttatgtggacacagatttcgattctatgtga

SEQ ID NO: 32 - protein, 687 aa, Saccharomyces cerevisiae
MTLKPSKRRKGRSRHSRKKQITSAILTEEGIMIKAKPSSPYTYANRMADKRSRSSIDNISRTSFQSNISRTSFQSNSDNNSIFETASLISCVTCLSDTDTIDRSETSTTDTSKDDLSANPKLHYPSVNGQLPANTVIPYGRILDARYIEKEPLHYYDANSSPSSPLSSSMSNISEKCDLDELESSQKKERKGNSLSRGSNSSSSLLTSRSPFTKLVEVIFARPRRHDVVPKRVSLYIDYKPHSSSHLKEEDDLVEEILKRSYKNTRRNKSIFVIINPFGGKGKAKKLFMTKAKPLLLASRCSIEVVYTKYPGHAIEIAREMDIDKYDTIACASGDGIPHEVINGLYQRPDHVKAFNNIAITEIPCGSGNAMSVSCHWTNNPSYSTLCLIKSIETRIDLMCCSQPSYAREHPKLSFLSQTYGLIAETDINTEFIRWMGPARFELGVAFNIIQKKKYPCEIYVKYAAKSKNELKNHYLEHKNKGSLEFQHITMNKDNEDCDNYNYENEYETENEDEDEDADADDEDSHLISRDLADSSADQIKEEDFKIKYPLDEGIPSDWERLDPNISNNLGIFYTGKMPYVAADTKFFPAALPSDGTMDMVITDARTSLTRMAPILLGLDKGSHVLQPEVLHSKILAYKIIPKLGNGLFSVDGEKFPLEPLQVEIMPRLCKTLLRNGRYVDTDFDSM

SEQ ID NO: 33 - DNA, 1038 bp, Saccharomyces cerevisiae
  source 1..1038: mol_type = genomic DNA
atgaacactaccacatctactgttatagcagcagttgccgaccagttccagtctttgaactcttcttcttcatgtttcttgaaggttcatgttccttccattgagaacccattcggtattgaattatggccaattttctccaaagtgtttgaatactttagtggctatccagctgagcaattcgagtttattcacaataagactttcttggctaacgggtatcatgctgttagtattattatcgtttattacattattatctttggtggccaagctatcttacgcgccttgaacgcctctccattaaagtttaaattgcttttcgagatacacaacttgtttttgacttctatttctctagttttatggttgctgatgttagaacagttggttcctatggtttatcacaacggtctattctggtctatctgctctaaggaagccttcgcaccaaaattagttactctttactatttgaactatttgaccaaattcgtagaattgattgacactgtgtttttagttttgagaagaaagaagttattgtttttgcacacttaccatcacggtgccaccgctttgttgtgctacactcaattaattggtcgtacttctgttgaatgggtagttatcctactaaacttgggtgttcacgttatcatgtactggtactacttcttgagttcatgtggtattagagtttggtggaagcaatgggtcactagattccaaattattcaatttttgattgacttggtatttgtttactttgctacctatacattctatgctcacaaatacttggacggtattttaccaaacaagggtacttgttatggtactcaggctgctgctgcttatgggtatttgattctaacatcttatttgcttttgtttatttccttctacatccaatcttacaagaaaggtggtaaaaagacagtcaagaaggaatctgaagtttccggctccgttgcatccggttcttctactggtgtcaagacctctaacaccaaggtctcttccaggaaagcttaa

SEQ ID NO: 34 - protein, 345 aa, Saccharomyces cerevisiae [sequence omitted]

SEQ ID NO: 35 - DNA, 1020 bp, Saccharomyces cerevisiae
  source 1..1020: mol_type = genomic DNA
atgccattacctccgtcaacattgaaccagaaatctaatagagtctactctgtagctagggtgtacaagaatgcctgcgaggagagaccacaagaatactgggactacgaacaaggggtgaccatcgattggggaaagatttccaattacgaaattatcaacaaaattggaagagggaaatattccgaagtgttcagcggtagatgtattgtaaacaaccagaagtgtgttattaaagttttaaaaccagttaaaatgaaaaaaatttatagagagttgaaaattctgaccaatctaacaggcggccccaatgttgttggcctttatgatatagtacaagacgctgactccaaaatacctgctttgatctttgaggaaatcaaaaatgttgatttcagaactttatatcctacattcaaacttcctgacatccagtattatttcacgcaattattgattgcgttagactactgtcactccatgggcataatgcacagagacgtaaagcctcagaatgtcatgattgatcctacggaacgtaaactaaggctgatcgattggggcctggcggagttctaccatccaggtgtagattacaacgttcgtgtcgcttcgcgttaccacaagggaccagaacttttagtaaacttgaaccaatatgactactccctagacttatggtcagtaggatgcatgctagcagctattgtcttcaaaaaagaaccttttttcaaagggtcgtctaatccagatcaactggtaaagattgccacagtactaggaaccaaggaactgttaggctatttgggtaagtacgggttgcacttaccatctgaatacgacaacattatgagagactttacaaaaaaatcgtggacacactttataacctccgagaccaaattagctgttcctgaagtggttgatttaatcgacaatttattaaggtatgaccatcaagaaagattaacagcaaaggaggctatggatcataagtttttcaaaacgaagtttgaataa

SEQ ID NO: 36 - protein, 339 aa, Saccharomyces cerevisiae
MPLPPSTLNQKSNRVYSVARVYKNACEERPQEYWDYEQGVTIDWGKISNYEIINKIGRGKYSEVFSGRCIVNNQKCVIKVLKPVKMKKIYRELKILTNLTGGPNVVGLYDIVQDADSKIPALIFEEIKNVDFRTLYPTFKLPDIQYYFTQLLIALDYCHSMGIMHRDVKPQNVMIDPTERKLRLIDWGLAEFYHPGVDYNVRVASRYHKGPELLVNLNQYDYSLDLWSVGCMLAAIVFKKEPFFKGSSNPDQLVKIATVLGTKELLGYLGKYGLHLPSEYDNIMRDFTKKSWTHFITSETKLAVPEVVDLIDNLLRYDHQERLTAKEAMDHKFFKTKFE

SEQ ID NO: 37 - DNA, 651 bp, Saccharomyces cerevisiae
  source 1..651: mol_type = genomic DNA
atgattgaccgcactaaaaacgaatctccagcttttgaagagtctccgcttacccccaatgtgtctaacctgaaaccattcccttctcaaagcaacaaaatatccactccagtgaccgaccataggagaagacggtcatccagcgtaatatcacatgtggaacaggaaaccttcgaagacgaaaatgaccagcagatgcttcccaacatgaacgctacgtgggtcgaccagcgaggcgcgtggttgattcatatcgtcgtaatagtactcttgaggctcttctactccttgttcgggtcgacgcccaaatggacgtggactttaacaaacatgacctacatcatcggattctatatcatgttccaccttgtcaaaggtacgcccttcgactttaacggtggtgcgtacgacaacctgaccatgtgggagcagattaacgatgagactttgtacacacccactagaaaatttctgctgattgtacccattgtgttgttcctgattagcaaccagtactaccgcaacgacatgacactattcctctccaacctcgccgtgacggtgcttattggtgtcgttcctaagctgggaattacgcatagactaagaatatccatccctggtattacgggccgtgctcaaattagttag

SEQ ID NO: 38 - protein, 216 aa, Saccharomyces cerevisiae
MIDRTKNESPAFEESPLTPNVSNLKPFPSQSNKISTPVTDHRRRRSSSVISHVEQETFEDENDQQMLPNMNATWVDQRGAWLIHIVVIVLLRLFYSLFGSTPKWTWTLTNMTYIIGFYIMFHLVKGTPFDFNGGAYDNLTMWEQINDETLYTPTRKFLLIVPIVLFLISNQYYRNDMTLFLSNLAVTVLIGVVPKLGITHRLRISIPGITGRAQIS

SEQ ID NO: 39 - DNA, 1083 bp, Saccharomyces cerevisiae
  source 1..1083: mol_type = genomic DNA
atgtcgatagtctacaataaaacaccattattacgtcaattcttccccggaaaggcttctgcacaatttttcttgaaatatgaatgccttcaaccaagtggctccttcaaaagtagaggaatcggtaatctcatcatgaaaagtgccattcgaattcaaaaggacggtaaaagatctcctcaggttttcgctagttctggcggtaatgccggttttgctgctgcaacagcatgtcaaagactgtctctaccatgtacagtcgtggttcctacagcgacaaagaagagaatggtagataaaatcaggaacaccggtgcccaggttatcgtgagtggtgcctactggaaagaagcagatacttttttaaaaacaaatgtcatgaataaaatagactctcaggtcattgagcccatttatgttcatcccttcgataatccggatatttgggaaggacattcatctatgatagatgaaatagtacaagatttgaaatcgcaacatatttccgtgaataaggttaaaggcatagtatgcagcgttggtggaggtggtttatacaatggtattattcaaggtttggaaaggtatggtttagctgataggatccctattgtgggggtggaaacgaatggatgtcatgttttcaatacttctttgaaaataggccaaccagttcaattcaagaagataacaagtattgctacttctctaggaacggccgtgatctctaatcaaactttcgaatacgctcgcaaatacaacaccagatccgttgtaatagaggacaaagatgttattgaaacctgtcttaaatatacacatcaattcaatatggtgattgaaccggcatgtggcgccgcattgcatttgggttacaacactaagatcctagaaaatgcactgggctcaaaattagctgcggatgacattgtgataattattgcttgtggcggctcctctaatactataaaggacttggaagaagcgttggatagcatgagaaaaaaagacactcctgtaatagaagtcgctgacaatttcatatttccagaaaaaaatattgtgaatttaaaaagtgcttga

SEQ ID NO: 40 - protein, 360 aa, Saccharomyces cerevisiae
MSIVYNKTPLLRQFFPGKASAQFFLKYECLQPSGSFKSRGIGNLIMKSAIRIQKDGKRSPQVFASSGGNAGFAAATACQRLSLPCTVVVPTATKKRMVDKIRNTGAQVIVSGAYWKEADTFLKTNVMNKIDSQVIEPIYVHPFDNPDIWEGHSSMIDEIVQDLKSQHISVNKVKGIVCSVGGGGLYNGIIQGLERYGLADRIPIVGVETNGCHVFNTSLKIGQPVQFKKITSIATSLGTAVISNQTFEYARKYNTRSVVIEDKDVIETCLKYTHQFNMVIEPACGAALHLGYNTKILENALGSKLAADDIVIIIACGGSSNTIKDLEEALDSMRKKDTPVIEVADNFIFPEKNIVNLKSA

SEQ ID NO: 41 - DNA, 65 bp, synthetic construct
  misc_feature 1..65: note = primer
  source 1..65: mol_type = other DNA
aagactataccattataaaaacgcataagaaacagtttcatcatgccagctgaagcttcgtacgc

SEQ ID NO: 42 - DNA, 67 bp, synthetic construct
  misc_feature 1..67: note = primer
  source 1..67: mol_type = other DNA
atatatatatatatatacatatatgcgtataggcagagccaactagcataggccactagtggatctg

SEQ ID NO: 43 - DNA, 65 bp, synthetic construct
  misc_feature 1..65: note = primer
  source 1..65: mol_type = other DNA
taagtgctggatagacaagagacaggaaaattaaccagcgagatgccagctgaagcttcgtacgc

SEQ ID NO: 44 - DNA, 67 bp, synthetic construct
  misc_feature 1..67: note = primer
  source 1..67: mol_type = other DNA
tcaagggcaaattgatgcttcaacgaaaaagttattggattttcagcataggccactagtggatctg

SEQ ID NO: 45 - DNA, 24 bp, synthetic construct
  misc_feature 1..24: note = primer
  source 1..24: mol_type = other DNA
ctgctctgtcaatatggcttcttg

SEQ ID NO: 46 - DNA, 45 bp, synthetic construct
  misc_feature 1..45: note = primer
  source 1..45: mol_type = other DNA
aaactcgaactgaaaaagcgtgtttgtaagaaatcatggcgcctc

SEQ ID NO: 47 - DNA, 72 bp, synthetic construct
  misc_feature 1..72: note = primer
  source 1..72: mol_type = other DNA
aatgccggaatcggtattgatttgggtaaaacctctgggatgtgtgccattttgtttgtttatgtgtgttta

SEQ ID NO: 48 - DNA, 22 bp, synthetic construct
  misc_feature 1..22: note = primer
  source 1..22: mol_type = other DNA
ctcctcaccttatcgtcatttc

SEQ ID NO: 49 - DNA, 48 bp, synthetic construct
  misc_feature 1..48: note = primer
  source 1..48: mol_type = other DNA
aaactcgaactgaaaaagcgtgtttggtgcggaaagactaagaaaaac

SEQ ID NO: 50 - DNA, 72 bp, synthetic construct
  misc_feature 1..72: note = primer
  source 1..72: mol_type = other DNA
aatgctagtggaaaggaaccggctgcagttgcattcgatgttacgttcattttgtttgtttatgtgtgttta

SEQ ID NO: 51 - DNA, 72 bp, synthetic construct
  misc_feature 1..72: note = primer
  source 1..72: mol_type = other DNA
aataagattgcctcaagaaaagaagttatcggtataatcttttgtaggtagcataggccactagtggatctg

SEQ ID NO: 52 - DNA, 75 bp, synthetic construct
  misc_feature 1..75: note = primer
  source 1..75: mol_type = other DNA
tgtgaaccaccagtgatcaacacaacttggtcttctaacgtaaacttcatgttgtttgtttatgtgtgtttattc

SEQ ID NO: 53 - DNA, 24 bp, synthetic construct
  misc_feature 1..24: note = primer
  source 1..24: mol_type = other DNA
gttaatacctgcacaattcaaccg

SEQ ID NO: 54 - DNA, 49 bp, synthetic construct
  misc_feature 1..49: note = primer
  source 1..49: mol_type = other DNA
aaactcgaactgaaaaagcgtgtttgtcttttatttcgatgtagagctg

SEQ ID NO: 55 - DNA, 72 bp, synthetic construct
  misc_feature 1..72: note = primer
  source 1..72: mol_type = other DNA
tcctctggttcgcacaggggcacacgggtatagtttgcaggagtactcattttgtttgtttatgtgtgttta

SEQ ID NO: 56 - DNA, 100 bp, synthetic construct
  misc_feature 1..100: note = primer
  source 1..100: mol_type = other DNA
agtggttgcagaggaggtcagtttgttgcatttgtaaaaaaagataataagaaaaatattcgtctagatttgagatccatgggcataatgcacagagacg

SEQ ID NO: 57 - DNA, 100 bp, synthetic construct
  misc_feature 1..100: note = primer
  source 1..100: mol_type = other DNA
ataaacgttattcgttgtgtgaaagttcttgaacgtgaaagaaagaaaaaagatgagacaaacaaggcaaccgtataggtgacaaggtcctcgaaaatag

SEQ ID NO: 58 - DNA, 100 bp, synthetic construct
  misc_feature 1..100: note = primer
  source 1..100: mol_type = other DNA
ctcatgcgattttattacccttgtttttcaattgaaaacccggaacgatttaacgagatcaagaactgttgaacaaaggtgacaaggtcctcgaaaatag

SEQ ID NO: 59 - DNA, 100 bp, synthetic construct
  misc_feature 1..100: note = primer
  source 1..100: mol_type = other DNA
gatacgtaaattctgtttccgaagatgcaagaaggagcagcagcaccagaaaaaattactatttttcttctccattccatgggcataatgcacagagacg

SEQ ID NO: 60 - DNA, 100 bp, synthetic construct
  misc_feature 1..100: note = primer
  source 1..100: mol_type = other DNA
tcagtttatgttgaatgccttctctccatgtttcaatgccagtatattcctgacatctgttgaatacctcatagccaggtgacaaggtcctcgaaaatag

SEQ ID NO: 61 - DNA, 100 bp, synthetic construct
  misc_feature 1..100: note = primer
  source 1..100: mol_type = other DNA
atgaatgccctaaaatatttctcaaatcatttaataactacaaagaaacaaaaaaaaatcaatgttgaggtgacaaccatgggcataatgcacagagacg

SEQ ID NO: 62 - DNA, 21 bp, synthetic construct
  misc_feature 1..21: note = primer
  source 1..21: mol_type = other DNA
agaacgaaggaaggagcacag

SEQ ID NO: 63 - DNA, 22 bp, synthetic construct
  misc_feature 1..22: note = primer
  source 1..22: mol_type = other DNA
tggcgaggtattggatagttcc

SEQ ID NO: 64 - DNA, 100 bp, synthetic construct
  misc_feature 1..100: note = primer
  source 1..100: mol_type = other DNA
actactctaccccatgacataaaaaatatctgtctccggttatatatgtctaaatagaattcgggtagcattttatcactagtggatctgatatcaccta

SEQ ID NO: 65 - DNA, 100 bp, synthetic construct
  misc_feature 1..100: note = primer
  source 1..100: mol_type = other DNA
tcaatagtggaagttgtttctccccaaacgccggggacagaactctctggatagttccaacgaaatattcccattttgtttgtttatgtgtgtttattcg

SEQ ID NO: 66 - DNA, 686 bp, Saccharomyces cerevisiae
  source 1..686: mol_type = genomic DNA
gatctcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacggacacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaa

SEQ ID NO: 67 - DNA, 499 bp, Saccharomyces cerevisiae
  source 1..499: mol_type = genomic DNA
accttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaacatgaatgtacggaggtagtttgctgattggcggtctatagataccttggttatggcgccctcacagccggcaggggaagcgcctacgcttgacatctactatatgtaagtatacggccc

SEQ ID NO: 68 - DNA, 750 bp, Saccharomyces cerevisiae
  source 1..750: mol_type = genomic DNA
acgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 69 - DNA, 532 bp, Saccharomyces cerevisiae
  source 1..532: mol_type = genomic DNA
agttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacacatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 70 - DNA, 326 bp, Saccharomyces cerevisiae
  source 1..326: mol_type = genomic DNA
ggcacgtccgacggcggcccgacgggtccgaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcatg

SEQ ID NO: 71 - DNA, 352 bp, Saccharomyces cerevisiae
  source 1..352: mol_type = genomic DNA
acaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaa

SEQ ID NO: 72 - DNA, 311 bp, Saccharomyces cerevisiae
  source 1..311: mol_type = genomic DNA
ggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatg

SEQ ID NO: 73 - DNA, 352 bp, Saccharomyces cerevisiae
  source 1..352: mol_type = genomic DNA
ataaagcactcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcaacgcg

SEQ ID NO: 74 - DNA, 351 bp, Saccharomyces cerevisiae
  source 1..351: mol_type = genomic DNA
agtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataactacatggatgataagaaaacatggagtacagtcactttgagaaccttcaatcagctggtaacgtcttcgttaattggatactcaaaaaagatggatagcatgaatcacaagatggaaggaaat

SEQ ID NO: 75 - DNA, 6979 bp, synthetic construct
  misc_feature 1..6979: note = plasmid
  misc_feature 2382: note = n is a, c, g, or t
  source 1..6979: mol_type = other DNA
agaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacaattccaaccttacccaagagttcgccaaactcagacatcactttangcaaaaccgcgccgtgcttcttcctcggtggcattcatcacgaaatgttcagcactacgcatacttttgacaggaaacgcaacggatattgagtcaatatcaggcattctatcgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccagggtacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatttaacactcagataatggttttaagtaaagtgtacaggatcggctctgcccctcgacggtatcgataagcttgatatcgaattcctgcagcccgggggatccactagttctagaatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtactagagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccta

SEQ ID NO: 76 - DNA, 3772 bp, synthetic construct
  misc_feature 1..3772: note = plasmid
  source 1..3772: mol_type = other DNA
gaacgcggccgccagctgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 77 - DNA, 4228 bp, synthetic construct
  misc_feature 1..4228: note = plasmid
  source 1..4228: mol_type = other DNA
gaacgcggccgccagctgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 78 - DNA, 4900 bp, synthetic construct
  misc_feature 1..4900: note = plasmid
  source 1..4900: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatccatggataaatgattatgttctaaacactcctcagaagctcatcgaactgtcatcctgcgtgaagattaaaatccaacttagaaatttcgagcttacggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcttacaaaaagatcacgtgatctgttgtattgaactgggatccgatatctagatgcattcgcgaggtaccgaggtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtgtgggatccgatatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 79 - DNA, 4860 bp, synthetic construct
  misc_feature 1..4860: note = plasmid
  source 1..4860: mol_type = other DNA
gtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaacctggcttatcgaaattaatacgactcactatagggagaccggcagatccgcggccgcataggccactagtggatctgatatcacctaataacttcgtatagcatacattatacgaagttatattaagggttctcgagagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacgggccctggaattcaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaacatgtgtgggatccgatatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatg

SEQ ID NO: 80 - DNA, 5960 bp, synthetic construct
  misc_feature 1..5960: note = plasmid
  source 1..5960: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtccatgggcataatgcacagagacgcgagatcatatcactgtggacgttgatgaaagaatacgttattctttcatcaaatcgttgctaaagaagctgccaccgccacggccaccgttgtagccgccgttgttgttattgtagttgctactgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcacggcccctagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgagaattaagaaaaagtcgtcatctttcgataagtttttcccacagcaaagcaatagtagaaaaaaacaatgggaaacgttgaatgaagacaaagcgtcgtggtttaaaaggaaatacgctcacgtacatgctagggaacaggaccgtgcagcggatccatatggcaagaaaaaggcccacgtcgagaagctaaaggagatcaagaatcaggcgaaattaaaccaaaagagccataaatccaagttccacgagatcatatcactgtggacgttgatgaaagaatacgttattctttcatcaaatcgtctattttcgaggaccttgtcacctagggggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 81 - DNA, 6391 bp, synthetic construct
  misc_feature 1..6391: note = plasmid
  source 1..6391: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtccatgggcataatgcacagagacgcgagatcatatcactgtggacgttgatgaaagaatacgttattctttcatcaaatcgttgctaaagaagctgccaccgccacggccaccgttgtagccgccgttgttgttattgtagttgctactgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttactttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgagaattaagaaaaagtcgtcatctttcgataagtttttcccacagcaaagcaatagtagaaaaaaacaatgggaaacgttgaatgaagacaaagcgtcgtggtttaaaaggaaatacgctcacgtacatgctagggaacaggaccgtgcagcggatccatatggcaagaaaaaggcccacgtcgagaagctaaaggagatcaagaatcaggcgaaattaaaccaaaagagccataaatccaagttccacgagatcatatcactgtggacgttgatgaaagaatacgttattctttcatcaaatcgtctattttcgaggaccttgtcacctagggggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 82 - DNA, 4114 bp, synthetic construct
  misc_feature 1..4114: note = plasmid
  source 1..4114: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccacaattaccggcagatccgcggccgcataggccactagtggatctgatatcacctaataacttcgtatagcatacattatacgaagttatattaagggttctcgagagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgcgtagatctggcctcgagatcccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 83 - DNA, 7807 bp, synthetic construct
  misc_feature 1..7807: note = plasmid
  source 1..7807: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaatattactacaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagctagtgtaatacgactcactatagggcgcggccgcagaattcgagctcggtacccgggatctcgaggccagatctactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggccgaccaagcgacgcccaacctgccatcacgagatttcgattccacggccgccttctatgaaaggttgggcttcggaatcgttttccgggacgccggctggatgatcctccagcgcggggatctcaagctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacccggacgggacgctcctgcgcctgatacagaacgaattgcttgcaggcatctcatgatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacaattccaaccttacccaagagttcgccaaactcagacatcactttaggcaaaaccgcgccgtgcttcttcctcggtggcattcatcacgaaatgttcagcactacgcatacttttgacaggaaacgcaacggatattgagtcaatatcaggcattctatcgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccagggtacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatgaattcaagctttgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaaatcacgagcgacggtaatttctttctcgataagaggccacgtgctttatgagggtaacatcaattcaagaaggagggaaacacttcctttttctggccctgataatagtatgagggtgaagccaaaataaaggattcgcgcccaaatcggcatctttaaatgcaggtatgcgatagttcctcactctttccttactcacgagtaattcttgcaaatgcctattatgcagatgttataatatctgtgcgtaattgtggatccgctctagagtcgacctgcaggcatgcaagcttgcggccgcgtattctatagtgtcacctaaatagcatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtttaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattataagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2023/0374558 A1
(43) Pub. Date: Nov. 23, 2023

(54) METHOD FOR PRODUCING PHYTOSPHINGOSINE OR PHYTOCERAMIDE

(71) Applicants: AJINOMOTO CO., INC., Tokyo (JP); EVOLVA SA, Reinach (CH)

(72) Inventors: Howard RIEZMAN, Avusy (CH); Sabina de Andrade Pereira TAVARES, Reinach (CH); Markus SCHWAB, Reinach (CH); Vsevolod Aleksandrovich SEREBRIANYI, Moscow (RU); Olga Aleksandrovna SOFYANOVICH, Moscow (RU); Anna Mikhailovna OZEROVA, Moscow (RU); Naoki IWANAGA, Kanagwa (JP)

(73) Assignees: AJINOMOTO CO., INC., Tokyo (JP); EVOLVA SA, Reinach (CH)

(21) Appl. No.: 18/350,982

(22) Filed: Jul. 12, 2023

Related U.S. Application Data
(63) Continuation of application No. PCT/JP2022/002034, filed on Jan. 20, 2022.

(30) Foreign Application Priority Data
Jan. 20, 2021 (RU) 2021101096

(51) Int. Cl. C12P 13/02; C12P 19/44; C12N 9/16; C12N 15/81

(52) U.S. Cl. CPC C12P 13/02; C12P 19/44; C12N 9/16; C12Y 301/03004; C12N 15/81; C12N 2800/102

(57) ABSTRACT

A method for producing an objective substance such as phytosphingosine (PHS) and phytoceramide (PHC) using yeast is provided. The objective substance is produced by cultivating yeast having an ability to produce the objective substance and modified so that the expression and/or activity of a protein(s) encoded by NEM1 and/or SPO7 gene(s) is reduced in a culture medium.

This application is a Continuation of, and claims priority under 35 U.S.C. § 120 to, International Application No. PCT/JP2022/002034, filed Jan. 20, 2022, and claims priority therethrough under 35 U.S.C. § 119 to Russian Patent Application No. 2021101096, filed Jan. 20, 2021, the entireties of which, as well as all citations cited herein, are incorporated by reference herein. The Sequence Listing filed herewith in ST.26 .xml format named 2023-07-12T_US-650_SEQ_LIST_st26.xml, 171,749 bytes, generated on Jun. 9, 2023 is also incorporated by reference.

### BACKGROUND

### Technical Field

The present invention relates to a method for producing an objective substance such as phytosphingosine (PHS) and phytoceramide (PHC) using yeast. PHS and PHC are industrially useful as ingredients for pharmaceuticals, cosmetics, and so forth.

### Background Art

Bioengineering techniques have been attempted to try to produce sphingoid bases and sphingolipids, such as PHS and PHC. Such Reported methods have included the use of yeast (see JP2014-529400, WO2017/033463, and WO2017/033464).

NEM1 and SPO7 genes encode, respectively, a catalytic subunit and a regulatory subunit of Nem1-Spo7 protein phosphatase (see Su W M, et. al., Yeast Nem1-Spo7 protein phosphatase activity on Pah1 phosphatidate phosphatase is specific for the Pho85-Pho80 protein kinase phosphorylation sites. J Biol Chem. 2014 Dec. 12; 289(50):34699-708). The Nem1-Spo7 protein phosphatase is a membrane-associated protein phosphatase complex and catalyzes the dephosphorylation of proteins such as phosphatidate phosphatase Pah1p. A relationship between the Nem1-Spo7 protein phosphatase and PHS or PHC production has not been previously reported.

### SUMMARY

An aspect of the present invention is the development of a novel technique for improving production of an objective substance such as phytosphingosine (PHS) and phytoceramide (PHC) by yeast and to provide a method for efficiently producing the objective substance.

The ability of yeast to produce an objective substance such as phytosphingosine (PHS) and phytoceramide (PHC) can be improved by modifying the yeast so that the expression and/or activity of a protein(s) encoded by NEM1 and/or SPO7 gene(s) is reduced.

It is an aspect of the present invention to provide a method for producing an objective substance, the method comprising cultivating yeast having an ability to produce the objective substance in a culture medium, wherein the yeast has been modified so that the expression and/or activity of a protein(s) encoded by NEM1 and/or SPO7 gene(s) is reduced as compared with a non-modified strain, and wherein the objective substance is selected from the group consisting of phytosphingosine (PHS) and phytoceramide (PHC).

It is a further aspect of the present invention to provide the method as described a above, wherein at least the expression and/or activity of the protein encoded by the NEM1 gene is reduced.

It is a further aspect of the present invention to provide the method as described a above, wherein the activity of each of the protein(s) is reduced by reducing the expression of the gene encoding the protein, or by disrupting the gene encoding the protein.

It is a further aspect of the present invention to provide the method as described a above, wherein the expression and/or activity of each of the protein(s) is reduced by deleting the gene encoding the protein.

It is a further aspect of the present invention to provide the method as described a above, wherein the protein encoded by the NEM1 gene is selected from the group consisting of: (A) a protein comprising the amino acid sequence of SEQ ID NO: 26; (B) a protein comprising the amino acid sequence of SEQ ID NO: 26 but including substitution, deletion, insertion, and/or addition of 1 to 10 amino acid residues, and functioning as a catalytic subunit of Nem1-Spo7 protein phosphatase; (C) a protein comprising an amino acid sequence having 90% or higher sequence identity to the amino acid sequence of SEQ ID NO: 26, and functioning as a catalytic subunit of Nem1-Spo7 protein phosphatase.

It is a further aspect of the present invention to provide the method as described above, wherein the protein encoded by the SPO7 gene is elected from the group consisting of: (A) a protein comprising the amino acid sequence of SEQ ID NO: 28; (B) a protein comprising the amino acid sequence of SEQ ID NO: 28 but including substitution, deletion, insertion, and/or addition of 1 to 10 amino acid residues, and functioning as a regulatory subunit of Nem1-Spo7 protein phosphatase; (C) a protein comprising an amino acid sequence having 90% or higher sequence identity to the amino acid sequence of SEQ ID NO: 28, and functioning as a regulatory subunit of Nem1-Spo7 protein phosphatase.

It is a further aspect of the present invention to provide the method as described above, wherein the objective substance is PHS, and the yeast has further been modified so that the expression and/or activity of one or more proteins encoded by a gene selected from the group consisting of LAG1, LAC1, LIP1, LCB4, LCB5, ELO3, CKA2, ORM2, CHA1, and combinations thereof is reduced as compared with a non-modified yeast, or wherein the objective substance is PHC, and the yeast has further been modified so that the expression and/or activity of one or more proteins encoded by a gene selected from the group consisting of YPC1, LCB4, LCB5, ORM2, CHA1, and combinations thereof is reduced as compared with a non-modified yeast.

It is a further aspect of the present invention to provide the method as described above, wherein the activity of each of the one or more proteins is reduced by reducing the expression of the gene encoding the protein, or by disrupting the gene encoding the protein.

It is a further aspect of the present invention to provide the method as described above, wherein the expression and/or activity of each of the one or more proteins is reduced by deletion of the gene encoding the protein.

It is a further aspect of the present invention to provide the method as described above, wherein the objective substance is PHS, and the yeast has further been modified so that the expression and/or activity of one or more proteins encoded by a gene selected from the group consisting of LCB1, LCB2, TSC10, SUR2, SER1, SER2, SER3, YPC1, and combinations thereof is increased as compared with a non-modified yeast, or wherein the objective substance is PHC, and the yeast has further been modified so that the expression and/or activity of one or more proteins encoded by a gene selected from the group consisting of LCB1, LCB2, TSC10, SUR2, LAG1, LAC1, LIP1, SER1, SER2, SER3, ELO3, and combinations thereof is increased as compared with a non-modified yeast.

It is a further aspect of the present invention to provide the method as described above, wherein at least the expression and/or activity of the protein encoded by the YPC1 gene is increased.

It is a further aspect of the present invention to provide the method as described above, wherein at least the expression and/or activity of one or more proteins encoded by a gene selected from the group consisting of SER1, SER2, SER3, and combinations thereof is increased.

It is a further aspect of the present invention to provide the method as described above, wherein the activity of each of the one or more proteins is increased by increasing the expression of the gene encoding the protein.

It is a further aspect of the present invention to provide the method as described above, wherein the expression and/or activity of each of the one or more proteins is increased by increasing the copy number of the gene encoding the protein, and/or by modifying an expression control sequence of the gene encoding the protein.

It is a further aspect of the present invention to provide the method as described above, wherein the PHS is selected from the group consisting of C16:0 PHS, C18:0 PHS, C20:0 PHS, C18:1 PHS, C20:1 PHS, 4-(hydroxymethyl)-2-methyl-6-tetradecanyl-1,3-oxazinan-5-ol, and 4-(hydroxymethyl)-2-methyl-6-hexadecanyl-1,3-oxazinan-5-ol.

It is a further aspect of the present invention to provide the method as described above, wherein the culture medium contains an additive that is able to associate with, bind to, solubilize, and/or capture the objective substance.

It is a further aspect of the present invention to provide the method as described above, wherein the additive is selected from the group consisting of cyclodextrin and zeolite.

It is a further aspect of the present invention to provide the method as described above, wherein the yeast belongs to the genus Saccharomyces.

It is a further aspect of the present invention to provide the method as described above, wherein the yeast is Saccharomyces cerevisiae.

It is a further aspect of the present invention to provide the method as described above, wherein the yeast is able to produce and accumulate the objective substance in a culture medium or cells of the yeast in an amount larger than that obtainable with a non-modified yeast.

It is a further aspect of the present invention to provide the method as described above, the method further comprising collecting the objective substance from cells of the yeast and/or the culture medium.

It is a further aspect of the present invention to provider a method for producing phytoceramide (PHC), the method comprising producing phytosphingosine (PHS) by the method as described above; and converting the PHS to the PHC.

### DETAILED DESCRIPTION OF EXEMPLARY EMBODIMENTS

The method as described herein is a method for producing an objective substance including the steps of cultivating yeast having an ability to produce the objective substance in a culture medium, wherein the yeast has been modified so that the expression and/or activity of a protein(s) encoded by NEM1 and/or SPO7 gene(s) is reduced. The yeast used for method is also referred to as “the yeast of the present invention”.

### <1> Yeast

The yeast as described herein is yeast having an ability to produce an objective substance, which has been modified so that the expression and/or activity of a protein(s) encoded by NEM1 and/or SPO7 gene(s) is reduced. The “ability to produce an objective substance” may also be referred to as “objective substance-producing ability”.

### <1-1> Yeast Having Objective Substance-Producing Ability

The phrase “yeast having an objective substance-producing ability” refers to yeast that is able to produce and accumulate an objective substance in a culture medium or cells of the yeast to such a degree that the objective substance can be collected, when the yeast is cultivated in the culture medium. The culture medium may be a culture medium that can be used in the method, and may specifically be a culture medium containing an additive that is able to associate with, bind to, solubilize, and/or capture the objective substance. The yeast having an objective substance-producing ability may also be yeast that is able to produce and accumulate an objective substance in a culture medium or cells of the yeast in an amount larger than that obtainable with a non-modified yeast strain. The term “non-modified yeast strain” may refer to a reference strain that has not been modified so that an objective substance-producing ability is imparted or enhanced, and may specifically refer to a reference strain that has not been modified so that the expression and/or activity of a protein(s) encoded by NEM1 and/or SPO7 gene(s) is reduced. Examples of the non-modified strain include a wild-type strain and parent strain, such as Saccharomyces cerevisiae strains BY4742 (ATCC 201389; EUROSCARF Y10000), S288C (ATCC 26108), and NCYC 3608. The yeast having an objective substance-producing ability may also be yeast that is able to produce and accumulate an objective substance in a culture medium in an amount of 5 mg/L or more, or 10 mg/L or more.

The objective substance can be either of phytosphingosine (PHS) or phytoceramide (PHC). Each variation of PHS is also referred to as “PHS species”. Each variation of PHC is also referred to as “PHC species”.

The term “phytosphingosine (PHS)” refers to a long-chain amino alcohol referred to as a sphingoid base, which has such a structure as described below. PHS has an alkyl chain having an amino group at C2. That is, the carbon present at either one terminus of the alkyl chain and linked to the aminated carbon (position C2) is regarded as position C1 of the alkyl chain. The alkyl chain has two or more hydroxyl groups. The alkyl chain may have hydroxyl groups, for example, at C1, C3, and C4. The alkyl chain may have or may not have additional hydroxyl group(s) other than the hydroxyl groups at C1, C3, and C4. The alkyl chain may typically have no additional hydroxyl group other than the hydroxyl groups at C1, C3, and C4. The configurations of chiral centers may or may not be identical to those in natural PHS species. The position C2 may be, for example, 2S. The position C3 may be, for example, 3S. The position C4 may be, for example, 4R. The configurations of chiral centers may be, for example, 2S, 3S, and 4R, which are typical configurations in natural PHS species. The length and the unsaturation degree of the alkyl chain may vary. The alkyl chain may have a length of, for example, C14 to C26, such as C14, C16, C18, C20, C22, C24, and C26. The alkyl chain may have a length of, for example, particularly, C16, C18, or C20. The length of the alkyl chain may be interpreted as the carbon number, i.e. the number of carbon atoms, of the alkyl chain. The alkyl chain may be saturated, or may be unsaturated. The alkyl chain may have one or more unsaturated double bonds. That is, the term “alkyl chain” does not be limited to saturated ones, but may also include unsaturated ones, such as alkenyl and alkadienyl chains, unless otherwise stated. The alkyl chain may typically have no or only one unsaturated double bond. The alkyl chain may more typically have no unsaturated double bond. The alkyl chain may have, for example, a C8-trans double bond. The number of carbons in the alkyl chain of PHS can be indicated as “n”. PHS having an alkyl chain of which the number of carbon is “n” is also referred to as “Cn PHS” or “Cn-alkyl PHS”. For example, the term “C18 PHS” collectively refers to PHS species having an alkyl chain having a length of C18, which may be saturated or unsaturated. The number of unsaturated double bonds in the alkyl chain of PHS can be indicated as “m”. PHS having an alkyl chain of which the number of carbon is “n” and the number of unsaturated double bond is “m” is also referred to as “Cn:m PHS” or “Cn:m-alkyl PHS”. Examples of PHS include such variant species of PHS, which variant species have different lengths and/or different unsaturation degrees. Specific examples of PHS include C16:0 PHS, which has a saturated C16 alkyl chain; C18:0 PHS, which has a saturated C18 alkyl chain; C20:0 PHS, which has a saturated C20 alkyl chain; C18:1 PHS, which has a C18 alkyl chain having one unsaturated double bond; and C20:1 PHS, which has a C20 alkyl chain having one unsaturated double bond. More specific examples of PHS include C16:0 PHS, C18:0 PHS, C20:0 PHS, C18:1 PHS, and C20:1 PHS, wherein each of them does not have any additional hydroxyl group other than the hydroxyl groups at C1, C3, and C4. Examples of PHS may also include adducts of PHS, such as 4-(hydroxymethyl)-2-methyl-6-tetradecanyl-1,3-oxazinan-5-ol and 4-(hydroxymethyl)-2-methyl-6-hexadecanyl-1,3-oxazinan-5-ol, which may be generated via reaction of C18:0 PHS and C20:0 PHS with acetaldehyde, respectively. The term “phytosphingosine (PHS)” is not limited to C18:0 PHS, which is a typical species of PHS, but may collectively refer to variant species of PHS, such as C16:0 PHS, C18:0 PHS, C20:0 PHS, C18:1 PHS, and C20:1 PHS, or may collectively refer to such variant species of PHS and adducts thereof. PHS may be a single kind of PHS species, or may be a mixture of two or more kinds of PHS species. Such a mixture may include two or more kinds of PHS species having different alkyl chains, such as alkyl chains having different lengths and/or different unsaturation degrees.

Phytoceramide (PHC) is a ceramide of phytosphingosine (PHS). PHC may also be referred to as, for example, “ceramide 3” or “ceramide NP”.

The term “phytoceramide (PHC)” refers to a compound including a structure of PHS covalently linked to a fatty acid via an amide bond. That is, PHC can include a PHS moiety, i.e. a moiety corresponding to PHS, and a fatty acid moiety, i.e. a moiety corresponding to a fatty acid, which moieties are covalently linked to each other via an amide bond. The PHS moiety can also be referred to as “alkyl chain”. The fatty acid moiety can also be referred to as “acyl chain”. The amide bond may form between the amino group at C2 of PHS and a carboxyl group of the fatty acid. The aforementioned descriptions concerning PHS can be applied similarly to the PHS moiety of PHC. That is, for example, the length and the unsaturation degree of the alkyl chain, i.e. the PHS moiety, may vary as with those of PHS. That is, examples of PHC include ceramides of the PHS species exemplified above. Specific examples of PHC include ceramides of C16 PHS, C18 PHS, and C20 PHS. More specific examples of PHC include ceramides of C16:0 PHS, C18:0 PHS, C20:0 PHS, C18:1 PHS, and C20:1 PHS. The length and the unsaturation degree of the acyl chain, i.e. the fatty acid moiety, of PHC may also vary. The acyl chain may have a length of, for example, C14 to C26, such as C14, C16, C18, C20, C22, C24, and C26. The acyl chain may have a length of, for example, particularly, C18. The length of the acyl chain may be interpreted as the carbon number, i.e. the number of carbon atoms, of the acyl chain. The acyl chain may be saturated, or may be unsaturated. The acyl chain may have one or more unsaturated double bonds. The acyl chain may or may not have a functional group, i.e. substituent group. The acyl chain may have one or more functional groups, such as substituent groups. Examples of the functional group can include a hydroxy group. The acyl chain may or may not have a hydroxy group, for example, at C2. The acyl chain may typically have no hydroxy group at C2. The carbon constituting the amide bond is regarded as position C1 of the acyl chain. The acyl chain may typically have no hydroxy group. The acyl chain may typically have no functional group. PHC having an alkyl chain of which the number of carbon is “n”, i.e. PHC having a Cn PHS moiety, is also referred to as “Cn alkyl PHC”, “(phyto)ceramide of Cn PHS”, or “Cn PHS (phyto)ceramide”. The number of carbons in the acyl chain of PHC can be indicated as “x”. PHC having an acyl chain of which the number of carbons is “x”, that is, PHC with a Cx acyl moiety, can also be referred to as “Cx acyl PHC”. The number of unsaturated double bonds in the acyl chain of PHC can be indicated as “y”. PHC having an acyl chain of which the number of carbons is “x” and the number of unsaturated double bonds is “y”, that is, PHC with a Cx:y acyl moiety, can also be referred to as “Cx:y acyl PHC”. PHC having an alkyl chain of which the number of carbons is “n” and an acyl chain of which the number of carbons is “x”, that is, PHC with a Cn PHS moiety and a Cx acyl moiety, can also be referred to as “Cn alkyl/Cx acyl PHC”. For example, the term “C18 alkyl PHC”, “ceramide of C18 PHS”, or “C18 PHS ceramide” collectively refers to PHC species having an alkyl chain having a length of C18 and having any acyl chain. For example, the term “C14 acyl PHC” collectively refers to a PHC species having an acyl chain with a length of C14 and having any alkyl chain. For example, the term “C18 alkyl/C14 acyl PHC” collectively refers to a PHC species having an alkyl chain with a length of C18 and an acyl chain with a length of C14. When PHC having an alkyl chain the number of carbons is “n” and the number of unsaturated double bonds is “m”, that is, PHC with a Cn:m PHS moiety, the term “Cn” used for the PHC name can be rewritten to “Cn:m”. The same shall apply to “Cx” of the acyl chain. For example, the term “C18:1 alkyl/C14:0 acyl PHC” collectively refers to a PHC species having an unsaturated alkyl chain with a length of C18 having one double bond and a saturated acyl chain with a length of C14. A PHC defined with the aforementioned name may be a single kind of PHC species, or may be a combination of two or more kinds of PHC species, unless otherwise stated. For example, a Cn PHS ceramide may be a single kind of Cn PHS ceramide, or may be a combination of two or more kinds of Cn PHS ceramides. Such a combination may be two or more kinds of Cn PHS ceramides having different alkyl chains and/or different acyl chains, such as alkyl chains having different unsaturation degrees and/or acyl chains having different lengths and/or different unsaturation degrees. Also, for example, a Cn:m PHS ceramide may be a single kind of Cn:m PHS ceramide, or may be a combination of two or more kinds of Cn:m PHS ceramides. Such a combination may be two or more kinds of Cn:m PHS ceramides having different acyl chains, such as acyl chains having different lengths and/or different unsaturation degrees. PHC may be a single kind of PHC species, or may be a mixture of two or more kinds of PHC species. Such a mixture may include two or more kinds of PHC species having different alkyl chains and/or different acyl chains, such as alkyl chains having different lengths and/or different unsaturation degrees and/or acyl chains having different lengths and/or different unsaturation degrees.

When the objective substance is a compound that can form a salt, the objective substance to be produced may be a free compound, a salt thereof, or a mixture thereof. That is, the term “objective substance” may refer to an objective substance in a free form, a salt thereof, or a mixture thereof, unless otherwise stated. Examples of the salt include, for example, inorganic acid salts such as sulfate salt, hydrochloride salt, and carbonate salt, and organic acid salts such as lactic acid salt and glycolic acid salt (Acta Derm Venereol. 2002; 82(3):170-3.). As the salt of the objective substance, a single kind of salt may be employed, or two or more kinds of salts may be employed.

The yeast is not particularly limited so long as it can be used for the method as described herein. The yeast may be budding yeast, or may be fission yeast. The yeast may be haploid yeast, or may be diploid or more polyploid yeast.

Examples of the yeast include yeast belonging to the genus Saccharomyces such as Saccharomyces cerevisiae, the genus Pichia (also referred to as the genus Wickerhamomyces) such as Pichia ciferrii, Pichia sydowiorum, and Pichia pastoris, the genus Candida such as Candida utilis, the genus Hansenula such as Hansenula polymorpha, the genus Schizosaccharomyces such as Schizosaccharomyces pombe. Some species of the genus Pichia has been reclassified into the genus Wickerhamomyces (Int J Syst Evol Microbiol. 2014 March; 64(Pt 3):1057-61). Therefore, for example, Pichia ciferrii and Pichia sydowiorum are also called Wickerhamomyces ciferrii and Wickerhamomyces sydowiorum, respectively. The term “Pichia” should include such species that had been classified into the genus Pichia but have been reclassified into another genus such as Wickerhamomyces.

Specific examples of Saccharomyces cerevisiae include strains BY4742 (ATCC 201389; EUROSCARF Y10000), S288C (ATCC 26108), Y006 (FERM BP-11299), NCYC 3608, and derivative strains thereof. Specific examples of Pichia ciferrii (Wickerhamomyces ciferrii) include strain NRRL Y-1031 (ATCC 14091), strain CS.PCΔPro2 (Schorsch et al., 2009, Curr Genet. 55, 381-9.), strains disclosed in WO 95/12683, and derivative strains thereof. Specific examples of Pichia sydowiorum (Wickerhamomyces sydowiorum) include strain NRRL Y-7130 (ATCC 58369) and derivative strains thereof.

These strains are available from, for example, the American Type Culture Collection (ATCC, Address: P.O. Box 1549, Manassas, VA 20108, United States of America), EUROpean Saccharomyces Cerevisiae ARchive for Functional Analysis (EUROSCARF, Address: Institute for Molecular Biosciences, Johann Wolfgang Goethe-University Frankfurt, Max-von-Laue Str. 9; Building N250, D-60438 Frankfurt, Germany), the National Collection of Yeast Cultures (NCYC, Address: Institute of Food Research, Norwich Research Park, Norwich, NR4 7UA, UK), or depositary institutions corresponding to deposited strains. That is, for example, in cases of ATCC strains, registration numbers are assigned to the respective strains, and the strains can be ordered by using these registration numbers (refer to www.atcc.org). The registration numbers of the strains are listed in the catalogue of the American Type Culture Collection (ATCC).

The yeast may inherently have an objective substance-producing ability, or may be modified so that it has an objective substance-producing ability. The yeast having an objective substance-producing ability can be obtained by imparting an objective substance-producing ability to yeast such as those described above, or by enhancing an objective substance-producing ability of yeast such as those described above.

Hereafter, methods for imparting or enhancing an objective substance-producing ability will be specifically exemplified. All the modifications for imparting or enhancing an objective substance-producing ability may be used independently or in any appropriate combination. Modifications for constructing the yeast can be performed in an arbitrary order.

An objective substance-producing ability may be imparted or enhanced by modifying yeast so that the expression and/or activity of one or more kinds of proteins involved in production of the objective substance are increased or reduced. That is, the yeast may have been modified so that the expression and/or activity of one or more kinds of proteins involved in production of the objective substance are increased or reduced. The term “protein” also includes so-called peptides such as polypeptides. Examples of the proteins involved in production of the objective substance include enzymes that catalyze the synthesis of the objective substance (also referred to as “biosynthetic enzyme of objective substance”), enzymes that catalyze a reaction branching away from the biosynthetic pathway of the objective substance to generate a compound other than the objective substance (also referred to as “biosynthetic enzyme of byproduct”), enzymes that catalyze decomposition of the objective substance (also referred to as “decomposition enzyme of objective substance”), proteins that affect, e.g. increase or reduce, the activity of an enzyme such as those described above.

The protein of which the expression and/or activity is to be increased or reduced can be appropriately chosen depending on the type of the objective substance and on the types and activities of the proteins involved in production of the objective substance and inherently possessed by the chosen yeast. For example, the expression and/or activity of one or more kinds of proteins such as biosynthetic enzymes of the objective substance may preferably be increased. Also, for example, the expression and/or activity of one or more kinds of proteins such as biosynthetic enzymes of a byproduct and decomposition enzymes of the objective substance may preferably be reduced.

Methods for increasing or reducing the expression and/or activity of a protein will be described in detail herein. The activity of a protein can be increased by, for example, increasing the expression of a gene encoding the protein. The activity of a protein can be reduced by, for example, reducing the expression of a gene encoding the protein or disrupting a gene encoding the protein. When increasing or reducing the expression and/or activity of two or more kinds of proteins, methods for increasing or reducing the expression and/or activity of each of the proteins can be independently chosen. The expression of a gene is also referred to as “the expression of a protein (i.e. the protein encoded by the gene)”. Such methods of increasing or reducing the expression and/or activity of a protein are well known in the art.

Specific examples of the proteins involved in production of the objective substance include proteins encoded by LCB1, LCB2, TSC10, SUR2, LAG1, LAC1, LIP1, SER1, SER2, SER3, YPC1, NEM1, SPO7, LCB4, LCB5, ELO3, CKA2, ORM2, and CHA1 genes. These genes may be collectively referred to as “target genes”, and proteins encoded thereby may be collectively referred to as “target proteins”.

The yeast at least has been modified so that the expression and/or activity of a protein(s) encoded by NEM1 and/or SPO7 gene(s) is reduced. The phrase “a protein(s) encoded by NEM1 and/or SPO7 gene(s)” refers to a protein encoded by NEM1 gene (Nem1p) and/or a protein encoded by SPO7 gene (Spo7p), i.e. either one or both of Nem1p and Spo7p. In particular, at least the expression and/or activity of Nem1p may be reduced. That is, for example, the expression and/or activity of Nem1p may be reduced, and the expression and/or activity of Spo7p may or may not further be reduced. The expression “the activity of a protein(s) encoded by NEM1 and/or SPO7 gene(s) is reduced” may specifically mean that the expression of NEM1 gene and/or SPO7 gene is reduced, or NEM1 gene and/or SPO7 gene is disrupted. Reduction in the expression and/or activity of a protein(s) encoded by NEM1 and/or SPO7 gene(s) results in an increased objective substance-producing ability, and thus results in an increased production of the objective substance. Examples of the increased production of the objective substance include an increased production amount of the objective substance, an increased production rate of the objective substance, and an increased yield of the objective substance. The yeast can be obtained by modifying yeast having an objective substance-producing ability so that the expression and/or activity of a protein(s) encoded by NEM1 and/or SPO7 gene(s) is reduced. The yeast can also be obtained by modifying yeast so that the expression and/or activity of a protein(s) encoded by NEM1 and/or SPO7 gene(s) is reduced, and then imparting or enhancing an objective substance-producing ability. The yeast may also be yeast that has acquired an objective substance-producing ability by being modified so that the expression and/or activity of a protein(s) encoded by NEM1 and/or SPO7 gene(s) is reduced.

The yeast may further have been modified so that the expression and/or activity of one or more proteins encoded by LCB1, LCB2, TSC10, SUR2, LAG1, LAC1, LIP1, SER1, SER2, SER3, YPC1, and ELO3 genes is increased, and/or that the expression and/or activity of one or more proteins encoded by LAG1, LAC1, LIP1, YPC1, LCB4, LCB5, ELO3, CKA2, ORM2, and CHA1 genes is reduced. The expression “the activity of one or more proteins encoded by LCB1, LCB2, TSC10, SUR2, LAG1, LAC1, LIP1, SER1, SER2, SER3, and YPC1 genes is increased” may specifically mean that the expression of one or more of the genes LCB1, LCB2, TSC10, SUR2, LAG1, LAC1, LIP1, SER1, SER2, SER3, and YPC1 is increased. The expression “the activity of one or more proteins encoded by YPC1, LCB4, LCB5, ELO3, CKA2, ORM2, and CHA1 genes is reduced” may specifically mean that the expression of one or more of the genes YPC1, LCB4, LCB5, ELO3, CKA2, ORM2, and CHA1 is reduced, or one or more of the genes YPC1, LCB4, LCB5, ELO3, CKA2, ORM2, and CHA1 are disrupted. The same shall apply similarly to other combinations of genes or proteins.

The yeast may have been modified so that the expression and/or activity of one or more proteins encoded by LCB1, LCB2, TSC10, SUR2, SER1, SER2, SER3, and YPC1 genes is increased, and/or that the expression and/or activity of one or more proteins encoded by LAG1, LAC1, LIP1, LCB4, LCB5, ELO3, CKA2, ORM2, and CHA1 genes is reduced, for example, when producing PHS. Alternatively, the yeast may have been modified so that the expression and/or activity of one or more proteins encoded by LCB1, LCB2, TSC10, SUR2, LAG1, LAC1, LIP1, SER1, SER2, SER3, and ELO3 genes is increased, and/or that the expression and/or activity of one or more proteins encoded by YPC1, LCB4, LCB5, ORM2, and CHA1 genes is reduced, for example, when producing PHC.

LCB1 and LCB2 genes encode serine palmitoyltransferase. The term “serine palmitoyltransferase” refers to a protein having an activity of catalyzing the synthesis of 3-ketosphinganine (3-ketodihydrosphingosine) from serine and palmitoyl-CoA (EC 2.3.1.50). This activity may be referred to as “serine palmitoyltransferase activity”. Proteins encoded by LCB1 and LCB2 genes may be referred to as “Lcb1p” and “Lcb2p”, respectively. Examples of LCB1 and LCB2 genes include those of yeast such as S. cerevisiae and Pichia ciferrii. The nucleotide sequences of LCB1 and LCB2 genes of S. cerevisiae S288C are shown as SEQ ID NOS: 1 and 3, and the amino acid sequences of Lcb1p and Lcb2p encoded thereby are shown as SEQ ID NOS: 2 and 4. Lcb1p and Lcb2p may form a heterodimer to function as serine palmitoyltransferase (Plant Cell. 2006 December; 18(12):3576-93.). The expression and/or activity of either one or both of Lcb1p and Lcb2p may be increased. An increased expression and/or activity of either one or both of Lcb1p and Lcb2p may specifically provide an increased serine palmitoyltransferase activity. Serine palmitoyltransferase activity can be measured by, for example, a known method (J Biol Chem. 2000 Mar. 17; 275(11):7597-603.).

TSC10 gene encodes 3-dehydrosphinganine reductase. The term “3-dehydrosphinganine reductase” refers to a protein having an activity of catalyzing the conversion of 3-ketosphinganine to dihydrosphingosine (DHS; sphinganine) in the presence of an electron donor such as NADPH (EC 1.1.1.102). This activity may be referred to as “3-dehydrosphinganine reductase activity”. A protein encoded by TSC10 gene may be referred to as “Tsc10p”. Examples of TSC10 gene include those of yeast such as S. cerevisiae and Pichia ciferrii. The nucleotide sequence of TSC10 gene of S. cerevisiae S288C is shown as SEQ ID NO: 5, and the amino acid sequence of Tsc10p encoded thereby is shown as SEQ ID NO: 6. An increased expression and/or activity of Tsc10p may specifically provide an increased 3-dehydrosphinganine reductase activity. 3-dehydrosphinganine reductase activity can be measured by, for example, a known method (Biochim Biophys Acta. 2006 January; 1761(1):52-63.).

SUR2 (SYR2) gene encodes sphingosine hydroxylase. The term “sphingosine hydroxylase” refers to a protein having an activity of catalyzing the hydroxylation of a sphingoid base or the hydroxylation of a sphingoid base moiety of a ceramide (EC 1.-.-.-). This activity may be referred to as “sphingosine hydroxylase activity”. Sphingosine hydroxylase may catalyze, for example, the hydroxylation of DHS to form PHS, or the hydroxylation of dihydroceramide, which is a ceramide of DHS, to form PHC. A protein encoded by SUR2 gene may be referred to as “Sur2p”. Examples of SUR2 gene include those of yeast such as S. cerevisiae and Pichia ciferrii. The nucleotide sequence of SUR2 gene of S. cerevisiae S288C is shown as SEQ ID NO: 7, and the amino acid sequence of Sur2p encoded thereby is shown as SEQ ID NO: 8. The nucleotide sequence of SUR2 gene of Pichia ciferrii is shown as SEQ ID NO: 9, and the amino acid sequence of Sur2p encoded thereby is shown as SEQ ID NO: 10. An increased expression and/or activity of Sur2p may specifically provide an increased sphingosine hydroxylase activity. Sphingosine hydroxylase activity can be measured by, for example, incubating the enzyme with DHS or a dihydroceramide and determining an enzyme-dependent production of PHS or PHC.

LAG1, LAC1, and LIP1 genes encode ceramide synthase. The term “ceramide synthase” refers to a protein having an activity of catalyzing the synthesis of a ceramide from a sphingoid base and an acyl-coenzyme A (EC 2.3.1.24). This activity may be referred to as “ceramide synthase activity”. Proteins encoded by LAG1, LAC1, and LIP1 genes may be referred to as “Lag1p”, “Lac1p”, and “Lip1p”, respectively. Examples of LAG1, LAC1, and LIP1 genes include those of yeast such as S. cerevisiae and Pichia ciferrii. The nucleotide sequences of LAG1, LAC1, and LIP1 genes of S. cerevisiae S288C are shown as SEQ ID NOS: 11, 13, and 15, and the amino acid sequences of Lag1p, Lac1p, and Lip1p encoded thereby are shown as SEQ ID NOS: 12, 14, and 16. LAG1 and LAC1 genes specifically encode functionally equivalent catalytic subunits of ceramide synthase. LIP1 gene specifically encodes a non-catalytic subunit of ceramide synthase. The non-catalytic subunit Lip1p is associated with each of the catalytic subunits Lag1p and Lac1p, and is required for ceramide synthase activity. The expression and/or activity of any one of Lag1p, Lac1p, and Lip1p may be increased alone, the expression and/or activity of either one of Lag1p and Lac1p may be increased in combination with Lip1p, the expression and/or activity of both of Lag1p and Lac1p may be increased, or the expression and/or activity of all of Lag1p, Lac1p, and Lip1p may be increased. The expression and/or activity of one or more of Lag1p, Lac1p, and Lip1p may be increased, for example, in cases of producing PHC. Alternatively, the expression and/or activity of one or more of Lag1p, Lac1p, and Lip1p may be reduced, for example, in cases of producing PHS. An increased or reduced expression and/or activity of one or more of Lag1p, Lac1p, and Lip1p may specifically provide an increased or reduced ceramide synthase activity. Ceramide synthase activity can be measured by, for example, a known method (Guillas, Kirchman, Chuard, Pfefferli, Jiang, Jazwinski and Conzelman (2001) EMBO J. 20, 2655-2665; Schorling, Vallee, Barz, Reizman and Oesterhelt (2001) Mol. Biol. Cell 12, 3417-3427; Vallee and Riezman (2005) EMBO J. 24, 730-741).

SER1, SER2, and SER3 genes encode L-serine biosynthesis enzymes. SER3 gene specifically encodes D-3-phosphoglycerate dehydrogenase. The term “D-3-phosphoglycerate dehydrogenase” refers to a protein having an activity of catalyzing the oxidation of 3-phosphoglycerate in the presence of an electron acceptor to form 3-phosphohydroxypyruvate (EC 1.1.1.95). This activity may be referred to as “D-3-phosphoglycerate dehydrogenase activity”. Examples of the electron acceptor include NADW. SER1 gene specifically encodes phosphoserine aminotransferase. The term “phosphoserine aminotransferase” refers to a protein having an activity of catalyzing the conversion of 3-phosphonooxypyruvate and L-glutamate to 0-phosphoserine and 2-oxoglutarate (EC 2.6.1.52). This activity may be referred to as “phosphoserine aminotransferase activity”. SER2 gene specifically encodes phosphoserine phosphatase. The term “phosphoserine phosphatase” refers to a protein having an activity of catalyzing the hydrolysis of O-phosphoserine to form serine (EC 3.1.3.3). This activity may be referred to as “phosphoserine phosphatase activity”. Proteins encoded by SER1, SER2, and SER3 genes may be referred to as “Ser1p”, “Ser2p”, and “Ser3p”, respectively. Examples of SER1, SER2, and SER3 genes include those of yeast such as S. cerevisiae and Pichia ciferrii. The nucleotide sequences of SER1, SER2, and SER3 genes of S. cerevisiae S288C are shown as SEQ ID NOS: 17, 19, and 21, and the amino acid sequences of Ser1p, Ser2p, and Ser3p encoded thereby are shown as SEQ ID NOS: 18, 20, and 22. The expression and/or activity of any one or more of Ser1p, Ser2p, and Ser3p may be increased. An increased expression and/or activity of Ser3p may specifically provide an increased D-3-phosphoglycerate dehydrogenase activity. An increased expression and/or activity of Ser1p may specifically provide an increased phosphoserine aminotransferase activity. An increased expression and/or activity of Ser2p may specifically provide an increased phosphoserine phosphatase activity. In addition, an increased expression and/or activity of one or more of Ser1p, Ser2p, and Ser3p may specifically provide an increased L-serine biosynthesis ability. D-3-phosphoglycerate dehydrogenase activity, phosphoserine aminotransferase activity, and phosphoserine phosphatase activity each can be measured by, for example, incubating the enzyme with the corresponding substrate and determining an enzyme-dependent production of the corresponding product.

YPC1 gene encodes phytoceramidase. The term “phytoceramidase” refers to a protein having an activity of catalyzing the decomposition of PHC (EC 3.5.1.-). This activity may be referred to as “phytoceramidase activity”. A protein encoded by YPC1 gene may be referred to as “Ypc1p”. Examples of YPC1 gene include those of yeast such as S. cerevisiae and Pichia ciferrii. The nucleotide sequence of YPC1 gene of S. cerevisiae S288C is shown as SEQ ID NO: 23, and the amino acid sequence of Ypc1p encoded thereby is shown as SEQ ID NO: 24. The expression and/or activity of Ypc1p may be increased, for example, in cases of producing PHS. Alternatively, the expression and/or activity of Ypc1p may be reduced, for example, in cases of producing PHC. An increased or reduced expression and/or activity of Ypc1p may specifically provide an increased or reduced phytoceramidase activity. Phytoceramidase activity can be measured by, for example, a known method (J Biol Chem. 2000 Mar. 10; 275(10):6876-84.).

NEM1 and SPO7 genes encode Nem1-Spo7 protein phosphatase. NEM1 and SPO7 genes specifically encode, respectively, a catalytic subunit and a regulatory subunit of Nem1-Spo7 protein phosphatase. The term “Nem1-Spo7 protein phosphatase” refers to a protein having an activity of catalyzing the dephosphorylation of protein, such as a phosphatidate phosphatase Pah1p. This activity may be referred to as “Nem1-Spo7 protein phosphatase activity”. Proteins encoded by NEM1 and SPO7 genes may be referred to as “Nem1p” and “Spo7p”, respectively. The nucleotide sequences of NEM1 and SPO7 genes of S. cerevisiae S288C are shown as SEQ ID NOS: 25 and 27, and the amino acid sequences of Nem1p and Spo7p encoded thereby are shown as SEQ ID NOS: 26 and 28. The expression and/or activity of either one or both of Nem1p and Spo7p may be reduced. A reduced expression and/or activity of either one or both of Nem1p and Spo7p may specifically provide a reduced Nem1-Spo7 protein phosphatase activity. Nem1-Spo7 protein phosphatase activity can be measured by, for example, a known method (Su W M, et. al., J Biol Chem. 2014 Dec. 12; 289(50):34699-708.).

LCB4 and LCB5 genes encode sphingoid base kinases. The term “sphingoid base kinase” refers to a protein having an activity of catalyzing the phosphorylation a sphingoid base to form a sphingoid base phosphate (EC 2.7.1.91). This activity may be referred to as “sphingoid base kinase activity”. Proteins encoded by LCB4 and LCB5 genes may be referred to as “Lcb4p” and “Lcb5p”, respectively. The nucleotide sequences of LCB4 and LCB5 genes of S. cerevisiae S288C are shown as SEQ ID NOS: 29 and 31, and the amino acid sequences of Lcb4p and Lcb5p encoded thereby are shown as SEQ ID NOS: 30 and 32. Of these, Lcb4p is the major sphingoid base kinase in S. cerevisiae (J Biol Chem. 2003 Feb. 28; 278(9):7325-34.). The expression and/or activity of either one or both of Lcb4p and Lcb5p may be reduced. At least the expression and/or activity of Lcb4p may be reduced. The activity of Lcb5p may also be reduced. A reduced expression and/or activity of either one or both of Lcb4p and Lcb5p may specifically provide a reduced sphingoid base kinase activity. Sphingoid base kinase activity can be measured by, for example, a known method (Plant Physiol. 2005 February; 137(2):724-37.).

ELO3 gene encodes fatty acid elongase III. The term “fatty acid elongase III” refers to a protein having an activity of catalyzing the elongation of C18-CoA to form C20-C26-CoA (EC 2.3.1.199). This activity may be referred to as “fatty acid elongase III activity”. C26-CoA may be used for the synthesis of ceramides catalyzed by ceramide synthase. A protein encoded by ELO3 gene may be referred to as “Elo3p”. The nucleotide sequence of ELO3 gene of S. cerevisiae S288C is shown as SEQ ID NO: 33, and the amino acid sequence of Elo3p encoded thereby is shown as SEQ ID NO: 34. The expression and/or activity of Elo3p may be increased, for example, in cases of producing PHC. Alternatively, the activity of Elo3p may be reduced, for example, in cases of producing PHS. An increased or reduced activity of Elo3p may specifically mean an increased or reduced fatty acid elongase III activity. Fatty acid elongase III activity can be measured by, for example, a known method (J Biol Chem. 1997 Jul. 11; 272(28):17376-84.).

CKA2 gene encodes an alpha′ subunit of casein kinase 2. The term “casein kinase 2” refers to a protein having an activity of catalyzing the serine/threonine-selective phosphorylation of proteins (EC 2.7.11.1). This activity may be referred to as “casein kinase 2 activity”. A protein encoded by CKA2 gene may be referred to as “Cka2p”. The nucleotide sequence of CKA2 gene of S. cerevisiae S288C is shown as SEQ ID NO: 35, and the amino acid sequence of Cka2p encoded thereby is shown as SEQ ID NO: 36. Cka2p may form a heterotetramer in combination with CKA1, CKB1, and CKB2 gene products, i.e. Cka1p, Ckb1p, and Ckb2p, to function as casein kinase 2. Cka2p may be required for full activation of ceramide synthase (Eukaryot Cell. 2003 April; 2(2):284-94.). The activity of Cka2p may be reduced, for example, in cases of producing PHS. A reduced activity of Cka2p may specifically mean a reduced casein kinase 2 activity. Also, a reduced activity of Cka2p may specifically mean a reduced ceramide synthase activity. Casein kinase 2 activity can be measured by, for example, a known method (Gene. 1997 Jun. 19; 192(2):245-50.).

ORM2 gene encodes a membrane protein that regulates serine palmitoyltransferase activity. A protein encoded by ORM2 gene may be referred to as “Orm2p”. The nucleotide sequence of ORM2 gene of S. cerevisiae S288C is shown as SEQ ID NO: 37, and the amino acid sequence of Orm2p encoded thereby is shown as SEQ ID NO: 38. A reduced activity of Orm2p may specifically mean an increased serine palmitoyltransferase activity.

CHA1 gene encodes L-serine/L-threonine ammonia-lyase. The term “L-serine/L-threonine ammonia-lyase” refers to a protein having an activity of catalyzing the decomposition of L-serine and L-threonine (EC 4.3.1.17 and EC 4.3.1.19). This activity may be referred to as “L-serine/L-threonine ammonia-lyase activity”. A protein encoded by CHA1 gene may be referred to as “Cha1p”. The nucleotide sequence of CHA1 gene of S. cerevisiae S288C is shown as SEQ ID NO: 39, and the amino acid sequence of Cha1p encoded thereby is shown as SEQ ID NO: 40. A reduced activity of Cha1p may specifically mean a reduced L-serine/L-threonine ammonia-lyase activity. L-serine/L-threonine ammonia-lyase activity can be measured by, for example, a known method (Eur J Biochem. 1982 April; 123(3):571-6.).

The target genes and proteins, i.e. LCB1, LCB2, TSC10, SUR2, LAG1, LAC1, LIP1, SER1, SER2, SER3, YPC1, NEM1, SPO7, LCB4, LCB5, ELO3, CKA2, ORM2, and CHA1 genes, and proteins encoded thereby, may have the aforementioned nucleotide and amino acid sequences. The expression “a gene or protein has a nucleotide or amino acid sequence” means that a gene or protein includes the nucleotide or amino acid sequence unless otherwise stated, and also includes cases where a gene or protein includes only the nucleotide or amino acid sequence.

The target genes may be variants of the respective genes exemplified above, so long as the original function thereof is maintained. Similarly, the target proteins may be variants of the respective proteins exemplified above, so long as the original function thereof is maintained. Such variants that maintain the original function thereof may also be referred to as “conservative variant”. A gene defined with the above-mentioned gene name or a protein defined with the above-mentioned protein name can include not only the genes or proteins of the same name exemplified above, respectively, but can also include conservative variants thereof. Namely, the terms “LCB1”, “LCB2”, “TSC10”, “SUR2”, “LAG1”, “LAC1”, “LIP1”, “SER1”, “SER2”, “SER3”, “YPC1”, “NEM1”, “SPO7”, “LCB4”, “LCB5”, “ELO3”, “CKA2”, “ORM2”, and “CHA1” genes include, in addition to the respective genes exemplified above, conservative variants thereof. Similarly, the terms “Lcb1p”, “Lcb2p”, “Tsc10p”, “Sur2p”, “Lag1p”, “Lac1p”, “Lip1p”, “Ser1p”, “Ser2p”, “Ser3p”, “Ypc1p”, “Nem1p”, “Spo7p”, “Lcb4p”, “Lcb5p”, “Elo3p”, “Cka2p”, “Orm2p”, and “Cha1p” include, in addition to the respective proteins exemplified above, conservative variants thereof. That is, for example, the term “LCB1 gene” includes the LCB1 gene exemplified above, e.g. LCB1 gene of S. cerevisiae, and further includes variants thereof. Similarly, for example, the term “Lcb1 protein” includes the Lcb1 protein exemplified above, e.g. the protein encoded by LCB1 gene of S. cerevisiae, and further includes variants thereof. Examples of the conservative variants include, for example, homologues and artificially modified versions of the target genes and proteins exemplified above. Methods of generating variants of a gene or a protein are well known in the art.

The expression “the original function is maintained” means that a variant of a gene or protein has a function, such as activity and property, corresponding to the function, such as activity and property, of the original gene or protein. The expression “the original function is maintained” regarding a gene means that a variant of the gene encodes a protein of which the original function is maintained. The expression “the original function is maintained” regarding a protein means that a variant of the protein has the corresponding function such as activity and property exemplified above. That is, the expression “the original function is maintained” regarding the target proteins may mean that a variant protein has serine palmitoyltransferase activity as for Lcb1p and Lcb2p; 3-dehydrosphinganine reductase activity as for Tsc10p; sphingosine hydroxylase activity as for Sur2p; ceramide synthase activity as for Lag1p, Lac1p, and Lip1p; D-3-phosphoglycerate dehydrogenase activity as for Ser3p; phosphoserine aminotransferase activity as for Ser1p; phosphoserine phosphatase activity as for Ser2p; phytoceramidase activity as for Ypc1p; Nem1-Spo7 protein phosphatase activity as for Nem1p and Spo7p; sphingoid base kinase activity as for Lcb4p and Lcb5p; fatty acid elongase III activity as for Elo3p; casein kinase 2 activity as for Cka2p; property of regulating serine palmitoyltransferase activity as for Orm2p; and L-serine/L-threonine ammonia-lyase activity as for Cha1p. The expression “the original function is maintained” regarding Nem1p may specifically mean that a variant of the protein has a function as a catalytic subunit of Nem1-Spo7 protein phosphatase. The expression “the original function is maintained” regarding Spo7p may specifically mean that a variant of the protein has a function as a regulatory subunit of Nem1-Spo7 protein phosphatase. In addition, the expression “the original function is maintained” regarding Cka2p may also mean that a variant of the protein has a property that a reduced activity thereof results in a reduced ceramide synthase activity. In addition, the expression “the original function is maintained” regarding Orm2p may also mean that a variant of the protein has a property that a reduced activity thereof results in an increased serine palmitoyltransferase activity. In cases where a target protein functions as a complex consisting of a plurality of subunits, the expression “the original function is maintained” regarding the target protein may also mean that a variant of the protein exhibits the corresponding function such as activity and property exemplified above in combination with other appropriate subunit(s). That is, for example, the expression “the original function is maintained” regarding Lcb1p may also mean that a variant protein has serine palmitoyltransferase activity in combination with an appropriate Lcb2p, and the expression “the original function is maintained” regarding Lcb2p may also mean that a variant protein has serine palmitoyltransferase activity in combination with an appropriate Lcb1p.

Hereafter, conservative variants will be described.

Homologues of the genes exemplified above or homologues of the proteins exemplified above can easily be obtained from a public database by, for example, BLAST search or FASTA search using the nucleotide sequence of any of the genes exemplified above or the amino acid sequence of any of the proteins exemplified above as a query sequence. Furthermore, homologues of the genes exemplified above can be obtained by, for example, PCR using the chromosome of an organism such as yeast as the template, and oligonucleotides prepared on the basis of the nucleotide sequence of any of the genes exemplified above as primers.

The target genes each may be a gene encoding a protein having any of the aforementioned amino acid sequences but including substitution, deletion, insertion, and/or addition of one or several amino acid residues at one or several positions, so long as the original function is maintained. For example, the encoded protein may have an extended or deleted N-terminus and/or C-terminus. Although the number meant by the term “one or several” used above may differ depending on the positions of amino acid residues in the three-dimensional structure of the protein or the types of amino acid residues, specifically, it is, for example, 1 to 50, 1 to 40, 1 to 30, 1 to 20, 1 to 10, 1 to 5, or 1 to 3.

The aforementioned substitution, deletion, insertion, and/or addition of one or several amino acid residues is a conservative mutation that maintains normal function of the protein. Typical examples of the conservative mutation are conservative substitutions. The conservative substitution is a mutation wherein substitution takes place mutually among Phe, Trp, and Tyr, if the substitution site is an aromatic amino acid; among Leu, Ile, and Val, if it is a hydrophobic amino acid; between Gln and Asn, if it is a polar amino acid; among Lys, Arg, and His, if it is a basic amino acid; between Asp and Glu, if it is an acidic amino acid; and between Ser and Thr, if it is an amino acid having a hydroxyl group. Examples of substitutions considered as conservative substitutions include, specifically, substitution of Ser or Thr for Ala, substitution of Gln, His, or Lys for Arg, substitution of Glu, Gln, Lys, His, or Asp for Asn, substitution of Asn, Glu, or Gln for Asp, substitution of Ser or Ala for Cys, substitution of Asn, Glu, Lys, His, Asp, or Arg for Gln, substitution of Gly, Asn, Gln, Lys, or Asp for Glu, substitution of Pro for Gly, substitution of Asn, Lys, Gln, Arg, or Tyr for His, substitution of Leu, Met, Val, or Phe for Ile, substitution of Ile, Met, Val, or Phe for Leu, substitution of Asn, Glu, Gln, His, or Arg for Lys, substitution of Ile, Leu, Val, or Phe for Met, substitution of Trp, Tyr, Met, Ile, or Leu for Phe, substitution of Thr or Ala for Ser, substitution of Ser or Ala for Thr, substitution of Phe or Tyr for Trp, substitution of His, Phe, or Trp for Tyr, and substitution of Met, Ile, or Leu for Val. Furthermore, such substitution, deletion, insertion, or addition of amino acid residues as mentioned above includes a naturally occurring mutation due to an individual difference, or a difference of species of the organism from which the gene is derived (mutant or variant).

Furthermore, the target genes each may be a gene encoding a protein having an amino acid sequence showing an identity of 80% or more, 90% or more, 95% or more, 97% or more, or 99% or more, to any of the total amino acid sequence mentioned above, so long as the original function is maintained.

Furthermore, the target genes each may be a DNA that is able to hybridize under stringent conditions with a probe that can be prepared from any of the aforementioned nucleotide sequences, such as a sequence complementary to the whole sequence or a partial sequence of any of the aforementioned nucleotide sequences, so long as the original function is maintained. The “stringent conditions” refer to conditions under which a so-called specific hybrid is formed, and a non-specific hybrid is not formed. Examples of the stringent conditions include those under which highly identical DNAs hybridize to each other, for example, DNAs not less than 80% identical, not less than 90% identical, not less than 95% identical, not less than 97% identical, or not less than 99% identical, hybridize to each other, and DNAs less identical than the above do not hybridize to each other, or conditions of washing of typical Southern hybridization, i.e., conditions of washing once, or 2 or 3 times, at a salt concentration and temperature corresponding to 1×SSC, 0.1% SDS at 60° C., 0.1×SSC, 0.1% SDS at 60° C., or 0.1×SSC, 0.1% SDS at 68° C.

The probe used for the aforementioned hybridization may be a part of a sequence that is complementary to the gene as described above. Such a probe can be prepared by PCR using oligonucleotides prepared on the basis of a known gene sequence as primers and a DNA fragment containing the nucleotide sequence as a template. As the probe, for example, a DNA fragment having a length of about 300 bp can be used. When a DNA fragment having a length of about 300 bp is used as the probe, in particular, the washing conditions of the hybridization may be, for example, 50° C., 2×SSC and 0.1% SDS.

Furthermore, the target genes each may be a gene having any of the aforementioned nucleotide sequences in which an arbitrary codon is replaced with an equivalent codon. That is, the target genes each may be a variant of any of the target genes exemplified above due to the degeneracy of the genetic code. For example, the target genes each may be a gene modified so that it has optimal codons according to codon frequencies in a host to be used.

The term “identity” between amino acid sequences means an identity between the amino acid sequences calculated by blastp with default scoring parameters (i.e. Matrix, BLOSUM62; Gap Costs, Existence=11, Extension=1; Compositional Adjustments, Conditional compositional score matrix adjustment). The term “identity” between nucleotide sequences means an identity between the nucleotide sequences calculated by blastn with default scoring parameters (i.e. Match/Mismatch Scores=1, −2; Gap Costs=Linear).

### <1-2> Methods for Increasing Activity of Protein

Hereafter, methods for increasing the activity of a protein will be explained.

The expression “the activity of a protein is increased” means that the activity of the protein is increased as compared with a non-modified strain. Specifically, the expression “the activity of a protein is increased” means that the activity of the protein per cell is increased as compared with that of a non-modified strain. The term “non-modified strain” may refer to a reference strain that has not been modified so that the activity of an objective protein is increased. Examples of the non-modified strain include a wild-type strain and parent strain. Specific examples of the non-modified strain include the respective type strains of the species of yeasts. That is, in an embodiment, the activity of a protein may be increased as compared with a type strain, i.e. the type strain of the species to which the yeast as described herein belongs. Specific examples of the non-modified strain also include the yeast strains described above, but prior to any modification. That is, in an embodiment, the activity of a protein may be increased as compared with a non-modified strain, which may be the same strain as that in which the protein is being increased but without the modification. In another embodiment, the activity of a protein may be increased as compared with Saccharomyces cerevisiae S288C (ATCC 26108). The state that “the activity of a protein is increased” may also be expressed as “the activity of a protein is enhanced”. More specifically, the expression “the activity of a protein is increased” may mean that the number of molecules of the protein per cell is increased, and/or the function of each molecule of the protein is increased as compared with those of a non-modified strain. That is, the term “activity” in the expression “the activity of a protein is increased” is not limited to the catalytic activity of the protein, but may also mean the transcription amount of a gene, i.e. the amount of mRNA, encoding the protein, or the translation amount of the gene, i.e. the amount of the protein. The term “the number of molecules of a protein per cell” may mean an average value of the number of molecules of the protein per cell. Although the degree of the increase in the activity of a protein is not particularly limited so long as the activity of the protein is increased as compared with that of a non-modified strain, the activity of the protein may be increased 1.5 times or more, 2 times or more, or 3 times or more, as compared with that of a non-modified strain. Furthermore, the state that “the activity of a protein is increased” includes not only a state that the activity of an objective protein is increased in a strain inherently having the activity of the objective protein, but also a state that the activity of an objective protein is imparted to a strain not inherently having the activity of the objective protein. Furthermore, so long as the activity of the protein is eventually increased, the activity of an objective protein inherently contained in a host may be attenuated and/or eliminated, and then an appropriate type of the objective protein may be introduced thereto.

The modification for increasing the activity of a protein is attained by, for example, increasing the expression of a gene encoding the protein. The expression “the expression of a gene is increased” means that the expression of the gene is increased as compared with that of a non-modified strain such as a wild-type strain and parent strain. Specifically, the expression “the expression of a gene is increased” means that the expression amount of the gene per cell is increased as compared with that of a non-modified strain. The term “the expression amount of a gene per cell” may mean an average value of the expression amount of the gene per cell. More specifically, the expression “the expression of a gene is increased” may mean that the transcription amount of the gene (i.e. the amount of mRNA) is increased, and/or the translation amount of the gene (i.e. the amount of the protein expressed from the gene) is increased. The state that “the expression of a gene is increased” may also be referred to as “the expression of a gene is enhanced”. The expression of a gene may be increased 1.5 times or more, 2 times or more, or 3 times or more, as compared with that observed in a non-modified strain. Furthermore, the state that “the expression of a gene is increased” includes not only a state that the expression amount of an objective gene is increased in a strain that inherently expresses the objective gene, but also a state that the gene is introduced into a strain that does not inherently express the objective gene, and expressed therein. That is, the phrase “the expression of a gene is increased” may also mean, for example, that an objective gene is introduced into a strain that does not possess the gene, and is expressed therein.

The expression of a gene can be increased by, for example, increasing the copy number of the gene.

The copy number of a gene can be increased by introducing the gene into the chromosome of a host. A gene can be introduced into a chromosome by, for example, using homologous recombination (Miller, J. H., Experiments in Molecular Genetics, 1972, Cold Spring Harbor Laboratory). Only one copy, or two or more copies of a gene may be introduced. For example, by performing homologous recombination using a sequence which is present in multiple copies on a chromosome as a target, multiple copies of a gene can be introduced into the chromosome. Examples of such a sequence which is present in multiple copies on a chromosome include autonomously replicating sequences (ARS) including a specific short repeated sequence, and rDNA sequences present in about 150 copies on the chromosome. WO95/32289 discloses an example where gene recombination was performed in yeast by using homologous recombination. In addition, a gene can also be introduced into a chromosome by, for example, integrating the gene into a transposon and transferring the transposon to the chromosome.

Introduction of an objective gene into a chromosome can be confirmed by Southern hybridization using a probe having a sequence complementary to the whole or a part of the gene, PCR using primers prepared on the basis of the sequence of the gene, or the like.

Furthermore, the copy number of an objective gene can also be increased by introducing a vector including the gene into a host. For example, the copy number of an objective gene can be increased by ligating a DNA fragment including the objective gene with a vector that functions in a host to construct an expression vector of the gene, and by transforming the host with the expression vector. The DNA fragment including the objective gene can be obtained by, for example, PCR using the genomic DNA of a microorganism having the objective gene as the template. As the vector, a vector autonomously replicable in the cell of the host can be used. The vector may be a single copy vector or may be a multi-copy vector. Furthermore, the vector includes a marker for selection of transformant. Examples of the marker include antibiotic resistance genes such as KanMX, NatMX (nat1), and HygMX (hph) genes, and genes complimenting auxotrophy such as LEU2, HIS3, and URA3 genes. Examples of vector autonomously replicable in yeast include plasmids having a CEN4 replication origin and plasmids having a 2 m DNA replication origin. Specific examples of vector autonomously replicable in yeast include pAUR123 (TAKARA BIO) and pYES2 (Invitrogen).

When a gene is introduced, it is sufficient that the gene is expressibly harbored by the chosen yeast. Specifically, it is sufficient that the gene is introduced so that it is expressed under the control of a promoter sequence that functions in the chosen yeast. The promoter may be a promoter derived from the host, or a heterogenous promoter. The promoter may be the native promoter of the gene to be introduced, or a promoter of another gene. As the promoter, for example, such a stronger promoter as mentioned later may also be used.

A terminator can be located downstream the gene. The terminator is not particularly limited as long as a terminator that functions in the yeast is chosen. The terminator may be a terminator derived from the host, or a heterogenous terminator. The terminator may be the native terminator of the gene to be introduced, or a terminator of another gene. Examples of the terminator that functions in the yeast as described herein include CYC1, ADH1, ADH2, ENO2, PGI1, and TDH1 terminators.

Vectors, promoters, and terminators available in various microorganisms are disclosed in detail in “Fundamental Microbiology Vol. 8, Genetic Engineering, KYORITSU SHUPPAN CO., LTD, 1987”, and those can be used.

Furthermore, when two or more kinds of genes are introduced, it is sufficient that the genes each are expressibly harbored by the yeast. For example, all the genes may be carried by a single expression vector or a chromosome. Alternatively, the genes may be separately carried by two or more expression vectors, or separately carried by a single or two or more expression vectors and a chromosome. An operon constituted by two or more genes may also be introduced.

The gene to be introduced is not particularly limited so long as it codes for a protein that functions in the host. The gene to be introduced may be a gene derived from the host, or may be a heterogenous gene. The gene to be introduced can be obtained by, for example, PCR using primers designed on the basis of the nucleotide sequence of the gene and the genomic DNA of an organism having the gene or a plasmid carrying the gene as a template. The gene to be introduced may also be totally synthesized, for example, on the basis of the nucleotide sequence of the gene (Gene, 60(1), 115-127 (1987)). The obtained gene can be used as it is, or after being modified as required.

Furthermore, the expression of a gene can be increased by improving the transcription efficiency of the gene. In addition, the expression of a gene can also be increased by improving the translation efficiency of the gene. The transcription efficiency of the gene and the translation efficiency of the gene can be improved by, for example, modifying an expression control sequence of the gene. The term “expression control sequence” collectively refers to sites that affect the expression of a gene, such as a promoter. Expression control sequences can be identified by using a promoter search vector or gene analysis software such as GENETYX.

The transcription efficiency of a gene can be improved by, for example, replacing the promoter of the gene on a chromosome with a stronger promoter. The “stronger promoter” means a promoter providing an improved transcription of a gene as compared with an inherently existing wild-type promoter of the gene. Examples of stronger promoters usable in yeast include PGK1, PGK2, PDC1, TDH3, TEF1, TEF2, TPI1, HXT7, ADH1, GPD1, and KEX2 promoters. Furthermore, as the stronger promoter, a highly-active type of an existing promoter may also be obtained by using various reporter genes. The translation efficiency of a gene can also be improved by, for example, modifying codons. For example, the translation efficiency of the gene can be improved by replacing a rare codon present in the gene with a more frequently used synonymous codon. That is, a gene to be introduced may have been modified, for example, so that it has optimal codons according to codon frequencies observed in the host to be used. Codons can be replaced by, for example, the site-specific mutation method for introducing an objective mutation into an objective site of DNA. Alternatively, a gene fragment in which objective codons are replaced may be totally synthesized. Frequencies of codons in various organisms are disclosed in the “Codon Usage Database” (www.kazusa.or.jp/codon; Nakamura, Y. et al, Nucl. Acids Res., 28, 292 (2000)).

Furthermore, the expression of a gene can also be increased by amplifying a regulator that increases the expression of the gene, or deleting or attenuating a regulator that reduces the expression of the gene.

Such methods for increasing the gene expression as mentioned above may be used independently or in an arbitrary combination.

Furthermore, the modification that increases the activity of an enzyme can also be attained by, for example, enhancing the specific activity of the enzyme. An enzyme showing an enhanced specific activity can be obtained by, for example, searching various organisms. Furthermore, a highly-active type of an existing enzyme may also be obtained by introducing a mutation into the existing enzyme. Enhancement of the specific activity may be independently used, or may be used in an arbitrary combination with such methods for enhancing the gene expression as mentioned above.

The method for transformation is not particularly limited, and methods conventionally used for transformation of yeast can be used. Examples of such methods include protoplast method, KU method (H. Ito et al., J. Bateriol., 153-163 (1983)), KUR method (Fermentation and industry, vol. 43, p. 630-637 (1985)), electroporation method (Luis et al., FEMS Micro biology Letters 165 (1998) 335-340), and a method using a carrier DNA (Gietz R. D. and Schiestl R. H., Methods Mol. Cell. Biol. 5:255-269 (1995)). Methods for manipulating yeast such as methods for spore-forming and methods for isolating haploid yeast are disclosed in Chemistry and Biology, Experimental Line 31, Experimental Techniques for Yeast, 1^{st} Edition, Hirokawa-Shoten; Bio-Manual Series 10, Genetic Experimental Methods for Yeast, 1^{st} Edition, Yodosha; and so forth.

An increase in the activity of a protein can be confirmed by measuring the activity of the protein.

An increase in the activity of a protein can also be confirmed by confirming an increase in the expression of a gene encoding the protein. An increase in the expression of a gene can be confirmed by confirming an increase in the transcription amount of the gene, or by confirming an increase in the amount of a protein expressed from the gene.

An increase of the transcription amount of a gene can be confirmed by comparing the amount of mRNA transcribed from the gene with that observed in a non-modified strain such as a wild-type strain or parent strain. Examples of the method for evaluating the amount of mRNA include Northern hybridization, RT-PCR, microarray, RNA-seq, and so forth (Sambrook, J., et al., Molecular Cloning A Laboratory Manual/Third Edition, Cold spring Harbor Laboratory Press, Cold Spring Harbor (USA), 2001). The amount of mRNA may increase, for example, 1.5 times or more, 2 times or more, or 3 times or more, as compared with that of a non-modified strain.

An increase in the amount of a protein can be confirmed by Western blotting using antibodies (Molecular Cloning, Cold Spring Harbor Laboratory Press, Cold Spring Harbor (USA), 2001). The amount of the protein may increase, for example, 1.5 times or more, 2 times or more, or 3 times or more, as compared with that of a non-modified strain.

### <1-3> Method for Reducing Activity of Protein

Hereafter, methods for reducing the activity of a protein will be explained.

The expression “the activity of a protein is reduced” means that the activity of the protein is reduced as compared with a non-modified strain. Specifically, the expression “the activity of a protein is reduced” means that the activity of the protein per cell is reduced as compared with that of a non-modified strain. The term “non-modified strain” may refer to a reference strain that has not been modified so that the activity of an objective protein is reduced. Examples of the non-modified strain include a wild-type strain or parent strain. Specific examples of the non-modified strain include the respective type strains of the species of yeasts. That is, in an embodiment, the activity of a protein may be reduced as compared with a type strain, i.e. the type strain of the species to which the yeast belongs. Specific examples of the non-modified strain also include the yeast strains described above, but prior to any modification. That is, in an embodiment, the activity of a protein may be reduced as compared with a non-modified strain, which may be the same strain as that in which the protein is being reduced but without the modification. In another embodiment, the activity of a protein may be reduced as compared with Saccharomyces cerevisiae S288C (ATCC 26108). The state that “the activity of a protein is reduced” also includes a state that the activity of the protein has completely disappeared. More specifically, the expression “the activity of a protein is reduced” may mean that the number of molecules of the protein per cell is reduced, and/or the function of each molecule of the protein is reduced as compared with those of a non-modified strain. That is, the term “activity” in the expression “the activity of a protein is reduced” is not limited to the catalytic activity of the protein, but may also mean the transcription amount of a gene (i.e. the amount of mRNA) encoding the protein or the translation amount of the gene (i.e. the amount of the protein). The term “the number of molecules of a protein per cell” may mean an average value of the number of molecules of the protein per cell. The state that “the number of molecules of the protein per cell is reduced” also includes a state that the protein does not exist at all. The state that “the function of each molecule of the protein is reduced” also includes a state that the function of each protein molecule completely disappears. Although the degree of the reduction in the activity of a protein is not particularly limited so long as the activity is reduced as compared with that of a non-modified strain, it may be reduced to, for example, 50% or less, 20% or less, 10% or less, 5% or less, or 0% of that of a non-modified strain.

The modification for reducing the activity of a protein can be attained by, for example, reducing the expression of a gene encoding the protein. The expression “the expression of a gene is reduced” means that the expression of the gene is reduced as compared with that of a non-modified strain such as a wild-type strain and parent strain. Specifically, the expression “the expression of a gene is reduced” means that the expression of the gene per cell is reduced as compared with that of a non-modified strain. The term “the expression amount of a gene per cell” may mean an average value of the expression amount of the gene per cell. More specifically, the expression “the expression of a gene is reduced” may mean that the transcription amount of the gene, i.e. the amount of mRNA, is reduced, and/or the translation amount of the gene, i.e. the amount of the protein expressed from the gene, is reduced. The state that “the expression of a gene is reduced” also includes a state that the gene is not expressed at all. The state that “the expression of a gene is reduced” is also referred to as “the expression of a gene is attenuated”. The expression of a gene may be reduced to 50% or less, 20% or less, 10% or less, 5% or less, or 0% of that of a non-modified strain.

The reduction in gene expression may be due to, for example, a reduction in the transcription efficiency, a reduction in the translation efficiency, or a combination of them. The expression of a gene can be reduced by modifying an expression control sequence of the gene such as a promoter. When an expression control sequence is modified, one or more nucleotides, two or more nucleotides, or three or more nucleotides, of the expression control sequence are modified. Furthermore, a part or the entire expression control sequence may be deleted. The expression of a gene can also be reduced by, for example, manipulating a factor responsible for expression control. Examples of the factor responsible for expression control include low molecules responsible for transcription or translation control (inducers, inhibitors, etc.), proteins responsible for transcription or translation control (transcription factors etc.), nucleic acids responsible for transcription or translation control (siRNA etc.), and so forth. Furthermore, the expression of a gene can also be reduced by, for example, introducing a mutation that reduces the expression of the gene into the coding region of the gene. For example, the expression of a gene can be reduced by replacing a codon in the coding region of the gene with a synonymous codon used less frequently in a host. Furthermore, for example, the gene expression may be reduced due to disruption of a gene as described herein.

The modification for reducing the activity of a protein can also be attained by, for example, disrupting a gene encoding the protein. The expression “a gene is disrupted” means that a gene is modified so that a protein that can normally function is not produced. The phrase that “a protein that normally functions is not produced” means when the protein is not produced at all from the gene, and also when the protein of which the function (such as activity or property) per molecule is reduced or eliminated is produced from the gene.

Disruption of a gene can be attained by, for example, deleting the gene on a chromosome. The term “deletion of a gene” refers to deletion of a partial or entire region of the coding region of the gene. Furthermore, the whole of a gene including sequences upstream and downstream from the coding region of the gene on a chromosome may be deleted. The region to be deleted may be any region such as an N-terminal region (region encoding an N-terminal region of a protein), an internal region, or a C-terminal region (region encoding a C-terminal region of a protein), so long as the activity of the protein can be reduced. Deletion of a longer region can usually more surely inactivate the gene. The region to be deleted may be, for example, a region having a length of 10% or more, 20% or more, 30% or more, 40% or more, 50% or more, 60% or more, 70% or more, 80% or more, 90% or more, or 95% or more of the total length of the coding region of the gene. Furthermore, it is preferred that reading frames of the sequences upstream and downstream from the region to be deleted are not the same. Inconsistency of reading frames may cause a frameshift downstream of the region to be deleted.

Disruption of a gene can also be attained by, for example, introducing a mutation for an amino acid substitution (missense mutation), a stop codon (nonsense mutation), addition or deletion of one or two nucleotide residues (frame shift mutation), or the like into the coding region of the gene on a chromosome (Journal of Biological Chemistry, 272:8611-8617 (1997); Proceedings of the National Academy of Sciences, USA, 95 5511-5515 (1998); Journal of Biological Chemistry, 26 116, 20833-20839 (1991)).

Disruption of a gene can also be attained by, for example, inserting another nucleotide sequence into a coding region of the gene on a chromosome. Site of the insertion may be in any region of the gene, and insertion of a longer nucleotide sequence can usually more surely inactivate the gene. It is preferred that reading frames of the sequences upstream and downstream from the insertion site are not the same. Inconsistency of reading frames may cause a frameshift downstream of the region to be deleted. The other nucleotide sequence is not particularly limited so long as a sequence that reduces or eliminates the activity of the encoded protein is chosen, and examples thereof include, for example, a marker gene such as antibiotic resistance genes, and a gene useful for production of an objective substance.

Particularly, disruption of a gene may be carried out so that the amino acid sequence of the encoded protein is deleted. In other words, the modification for reducing the activity of a protein can be attained by, for example, deleting the amino acid sequence of the protein, specifically, modifying a gene so as to encode a protein of which the amino acid sequence is deleted. The term “deletion of the amino acid sequence of a protein” refers to deletion of a partial or entire region of the amino acid sequence of the protein. In addition, the term “deletion of the amino acid sequence of a protein” means that the original amino acid sequence disappears in the protein, and also includes cases where the original amino acid sequence is changed to another amino acid sequence. That is, for example, a region that was changed to another amino acid sequence by frameshift may be regarded as a deleted region. When the amino acid sequence of a protein is deleted, the total length of the protein is typically shortened, but there can also be cases where the total length of the protein is not changed or is extended. For example, by deletion of a partial or entire region of the coding region of a gene, a region encoded by the deleted region can be deleted in the encoded protein. In addition, for example, by introduction of a stop codon into the coding region of a gene, a region encoded by the downstream region of the introduction site can be deleted in the encoded protein. In addition, for example, by frameshift in the coding region of a gene, a region encoded by the frameshift region can be deleted in the encoded protein. The aforementioned descriptions concerning the position and length of the region to be deleted in deletion of a gene can be applied similarly to the position and length of the region to be deleted in deletion of the amino acid sequence of a protein.

Such modification of a gene on a chromosome as described above can be attained by, for example, homologous recombination using a recombinant DNA. The structure of the recombinant DNA to be used for homologous recombination is not particularly limited as long as it causes homologous recombination in a desired manner. For example, a host can be transformed with a linear DNA including an arbitrary sequence such as a disruption-type gene or any appropriate insertion sequence, which arbitrary sequence is flanked with upstream and downstream sequences of the homologous recombination target region on the chromosome, so that homologous recombination can occur at upstream and downstream sides of the target region, to thereby replace the target region with the arbitrary sequence. Specifically, such modification of a gene on a chromosome as described above can be attained by, for example, preparing a disruption-type gene modified so that it cannot produce a protein that can normally function, and transforming a host with a recombinant DNA including the disruption-type gene to cause homologous recombination between the disruption-type gene and the wild-type gene on a chromosome and thereby substitute the disruption-type gene for the wild-type gene on the chromosome. In this procedure, if a marker gene selected according to the characteristics of the host such as auxotrophy is included in the recombinant DNA, the operation becomes easy. Examples of the disruption-type gene include a gene in which a part or whole of the gene is deleted, a gene introduced with missense mutation, a gene introduced with nonsense mutation, a gene introduced with frameshift mutation, and a gene introduced with an insertion sequence such as a transposon and a marker gene. The protein encoded by the disruption-type gene has a conformation different from that of the wild-type protein, even if it is produced, and thus the function thereof is reduced or eliminated. The modification for reducing the activity of a protein can also be attained by, for example, a mutagenesis treatment. Examples of the mutagenesis treatment include usual mutation treatments such as irradiation of X-ray or ultraviolet and treatment with a mutation agent such as N-methyl-N′-nitro-N-nitrosoguanidine (MNNG), ethyl methanesulfonate (EMS), and methyl methanesulfonate (MMS).

Such methods for reducing the activity of a protein as mentioned above may be used independently or in an arbitrary combination.

A reduction in the activity of a protein can be confirmed by measuring the activity of the protein.

A reduction in the activity of a protein can also be confirmed by confirming a reduction in the expression of a gene encoding the protein. A reduction in the expression of a gene can be confirmed by confirming a reduction in the transcription amount of the gene or a reduction in the amount of the protein expressed from the gene.

A reduction in the transcription amount of a gene can be confirmed by comparing the amount of mRNA transcribed from the gene with that observed in a non-modified strain. Examples of the method for evaluating the amount of mRNA include Northern hybridization, RT-PCR, microarray, RNA-seq, and so forth (Molecular Cloning, Cold spring Harbor Laboratory Press, Cold Spring Harbor (USA), 2001). The amount of mRNA can be reduced to, for example, 50% or less, 20% or less, 10% or less, 5% or less, or 0%, of that observed in a non-modified strain.

A reduction in the amount of a protein can be confirmed by Western blotting using antibodies (Molecular Cloning, Cold Spring Harbor Laboratory Press, Cold Spring Harbor (USA) 2001). The amount of the protein can be reduced to, for example, 50% or less, 20% or less, 10% or less, 5% or less, or 0%, of that observed in a non-modified strain.

Disruption of a gene can be confirmed by determining nucleotide sequence of a part or the whole of the gene, restriction enzyme map, full length, or the like of the gene depending on the means used for the disruption.

### <2> Method

The method as described herein is a method for producing an objective substance including steps of cultivating the yeast as described herein in a culture medium. In the method, a single kind of objective substance may be produced, or two or more kinds of objective substances may be produced.

The culture medium to be used is not particularly limited, so long as the chosen yeast can proliferate in it, and an objective substance can be produced. As the culture medium, for example, a usual culture medium used for cultivating yeast can be used. Examples of such a culture medium include SD medium, SG medium, SDTE medium, and YPD medium. The culture medium may contain a carbon source, nitrogen source, phosphorus source, and sulfur source, as well as components selected from other various organic components and inorganic components as required. The types and concentrations of the culture medium components can be appropriately determined according to various conditions such as the type of the yeast to be used and the type of the objective substance to be produced.

The culture medium may contain an additive that is able to associate with, bind to, solubilize, and/or capture the objective substance (WO2017/033463). Use of the additive may result in an increased production of the objective substance. That is, the amount produced of the objective substance by the yeast may be increased in the presence of the additive as compared with in the absence of the additive. Use of the additive may specifically result in an increased production of the objective substance in the culture medium. The production of the objective substance in the culture medium may also be referred to as “excretion of the objective substance”. The expression “associating with, binding to, solubilizing, and/or capturing an objective substance” may specifically mean increasing the solubility of the objective substance into the culture medium. Examples of the additive include cyclodextrins and zeolites. The number of glucose residues constituting cyclodextrins is not particularly limited, and it may be, for example, 5, 6, 7, or 8. That is, examples of cyclodextrins include cyclodextrin consisting of 5 glucose residues, alpha-cyclodextrin, beta-cyclodextrin, gamma-cyclodextrin, and derivatives thereof. Examples of cyclodextrin derivatives include cyclodextrins into which one or more functional groups have been introduced. The type, number, and amount of the functional group, and the position to which the functional group is introduced are not particularly limited as long as the derivative is able to associate with, bind to, solubilize, and/or capture the objective substance. The functional group may be introduced to, for example, hydroxyl group of C2, C3, C6, or a combination thereof, which may result in an increased solubility of cyclodextrin itself. Examples of the functional group include alkyl groups and hydroxyalkyl groups. The alkyl groups and hydroxyalkyl groups each may have a linear alkyl chain or may have a branched alkyl chain. The alkyl groups and hydroxyalkyl groups each may have a carbon number of, for example, 1, 2, 3, 4, or 5. Specific examples of the alkyl groups include methyl, ethyl, propyl, butyl, pentyl, isopropyl, and isobutyl groups. Specific examples of the hydroxyalkyl groups include hydroxymethyl, hydroxyethyl, hydroxypropyl, hydroxybutyl, hydroxypentyl, hydroxyisopropyl, and hydroxyisobutyl groups. Specific examples of cyclodextrin derivatives include methyl-alpha-cyclodextrin, methyl-beta-cyclodextrin, hydroxypropyl-alpha-cyclodextrin such as 2-hydroxypropyl-alpha-cyclodextrin, and hydroxypropyl-beta-cyclodextrin such as 2-hydroxypropyl-beta-cyclodextrin. The types of zeolites are not particularly limited. As the additive, a single kind of additive may be used, or two or more kinds of additives may be used in combination.

The additive may be present in the culture medium over the whole period of the culture, or may be present in the culture medium during only a partial period of the culture. That is, the phrase “cultivating yeast in a culture medium containing an additive” does not necessarily mean that the additive is present in the culture medium over the whole period of the culture. For example, the additive may be or may not be present in the culture medium from the start of the culture. When the additive is not present in the culture medium at the time of the start of the culture, the additive is supplied to the culture medium after the start of the culture. Timing of the supply can be appropriately determined according to various conditions such as the length of culture period. For example, the additive may be supplied to the culture medium after the yeast fully grows. Furthermore, in any case, the additive may be additionally supplied to the culture medium as required. Means for supplying the additive to the culture medium is not particularly limited. For example, the additive can be supplied to the culture medium by feeding a feed medium containing the additive to the culture medium. The concentration of the additive in the culture medium is not particularly limited so long as the objective substance can be produced. For example, the concentration of the additive in the culture medium may be 0.1 g/L or higher, 1 g/L or higher, 2 g/L or higher, 5 g/L or higher, or 10 g/L or higher, may be 200 g/L or lower, 100 g/L or lower, 50 g/L or lower, or 20 g/L or lower, or may be within a range defined with a combination thereof. The concentration of the additive in the culture medium may be, for example, 0.1 g/L to 200 g/L, 1 g/L to 100 g/L, or 5 g/L to 50 g/L. The additive may be or may not be present in the culture medium at a concentration within the range exemplified above during the whole period of the culture. For example, the additive may be contained in the culture medium at a concentration within the range exemplified above at the start of the culture, or it may be supplied to the culture medium so that a concentration within the range exemplified above is attained after the start of the culture.

Specific examples of the carbon source include, for example, saccharides such as glucose, fructose, sucrose, lactose, galactose, xylose, arabinose, blackstrap molasses, starch hydrolysates, and hydrolysates of biomass, organic acids such as acetic acid, fumaric acid, citric acid, and succinic acid, alcohols such as glycerol, crude glycerol, and ethanol, and fatty acids. As the carbon source, a single kind of carbon source may be used, or two or more kinds of carbon sources may be used in combination.

Specific examples of the nitrogen source include, for example, ammonium salts such as ammonium sulfate, ammonium chloride, and ammonium phosphate, organic nitrogen sources such as peptone, yeast extract, meat extract, and soybean protein decomposition products, ammonia, and urea. Ammonia gas or aqueous ammonia used for adjusting pH may also be used as the nitrogen source. As the nitrogen source, a single kind of nitrogen source may be used, or two or more kinds of nitrogen sources may be used in combination.

Specific examples of the phosphate source include, for example, phosphoric acid salts such as potassium dihydrogenphosphate and dipotassium hydrogenphosphate, and phosphoric acid polymers such as pyrophosphoric acid. As the phosphate source, a single kind of phosphate source may be used, or two or more kinds of phosphate sources may be used in combination.

Specific examples of the sulfur source include, for example, inorganic sulfur compounds such as sulfates, thiosulfates, and sulfites, and sulfur-containing amino acids such as cysteine, cystine, and glutathione. As the sulfur source, a single kind of sulfur source may be used, or two or more kinds of sulfur sources may be used in combination.

Specific examples of other various organic components and inorganic components include, for example, inorganic salts such as sodium chloride and potassium chloride; trace metals such as iron, manganese, magnesium, and calcium; vitamins such as vitamin B1, vitamin B2, vitamin B6, nicotinic acid, nicotinamide, and vitamin B12; amino acids; nucleic acids; and organic components containing those such as peptone, casamino acid, yeast extract, and soybean protein decomposition product. As other various organic components and inorganic components, a single kind of component may be used, or two or more kinds of components may be used in combination.

Furthermore, when an auxotrophic mutant that requires an amino acid, a nucleic acid, or the like for growth thereof is used, it is preferable to supplement a required nutrient to the culture medium.

The culture conditions are not particularly limited so long as the chosen yeast can proliferate, and the objective substance can be produced. The culture can be performed, for example, under usual conditions used for cultivating yeast. The culture conditions can be appropriately determined according to various conditions such as the type of yeast to be used and the type of objective substance to be produced.

The culture can be performed by using a liquid medium under an aerobic condition, a microaerobic condition, or an anaerobic condition. The culture can be performed under an aerobic condition. The term “aerobic condition” may refer to a condition where the dissolved oxygen concentration in the liquid medium is 0.33 ppm or higher, or 1.5 ppm or higher. In cases of the aerobic condition, the oxygen concentration can be controlled to be, for example, 5 to 50%, about 10 to 20%, of the saturated oxygen concentration. Specifically, the aerobic culture can be performed with aeration or shaking. The term “microaerobic condition” may refer to a condition where oxygen is supplied to the culture system but the dissolved oxygen concentration in the liquid medium is lower than 0.33 ppm. The term “anaerobic condition” may refer to a condition where oxygen is not supplied to the culture system. The culture temperature may be, for example, 25 to 35° C., 27 to 33° C., or 28 to 32° C. The pH of the culture medium may be, for example, 3 to 10, or 4 to 8. The pH of the culture medium may be adjusted as required during the culture. For adjusting pH, inorganic or organic acidic or alkaline substances, such as ammonia gas and so forth, can be used. The culture period may be, for example, 10 to 200 hours, or 15 to 120 hours. The culture condition may be constant during the whole period of the culture, or may be changed during the culture. The culture can be performed as batch culture, fed-batch culture, continuous culture, or a combination of these. Furthermore, the culture may be performed as two steps of a seed culture and a main culture. In such a case, the culture conditions of the seed culture and the main culture may or may not be the same. For example, both the seed culture and the main culture may be performed as batch culture. Alternatively, for example, the seed culture may be performed as batch culture, and the main culture may be performed as fed-batch culture or continuous culture.

By culturing the yeast under such conditions, the objective substance is accumulated in the culture medium and/or cells of the yeast.

Production of the objective substance can be confirmed by known methods used for detection or identification of compounds. Examples of such methods include, for example, HPLC, UPLC, LC/MS, GC/MS, and NMR. These methods may be used independently or in any appropriate combination.

The produced objective substance can be appropriately collected. That is, the method may further include a step of collecting the objective substance from cells of the yeast and/or the culture medium. The produced objective substance can be collected by known methods used for separation and purification of compounds. Examples of such methods include, for example, ion-exchange resin method, membrane treatment, precipitation, and crystallization. These methods may be used independently or in any appropriate combination. When the objective substance accumulates in cells, the cells can be disrupted with, for example, ultrasonic waves or the like, and then the objective substance can be collected from the supernatant obtained by removing the cells from the cell-disrupted suspension by centrifugation. The objective substance to be collected may be a free compound, a salt thereof, or a mixture thereof.

Furthermore, when the objective substance deposits in the culture medium, it can be collected by centrifugation, filtration, or the like. The objective substance deposited in the culture medium may also be isolated together with the objective substance dissolved in the culture medium after the objective substance dissolved in the culture medium is crystallized.

The objective substance collected may contain additional substance(s) such as yeast cells, culture medium components, moisture, and by-product metabolites of the yeast, in addition to the objective substance. The purity of the objective substance collected may be, for example, 30% (w/w) or higher, 50% (w/w) or higher, 70% (w/w) or higher, 80% (w/w) or higher, 90% (w/w) or higher, or 95% (w/w) or higher.

When PHS is produced by cultivation of the yeast, the thus-produced PHS can be converted to a corresponding PHC. Described herein is a method for producing PHC, the method including a step of producing PHS by the method as described herein, and converting the PHS to the PHC.

PHS produced by cultivation of the yeast can be used for the conversion to PHC as it is, or after being subjected to an appropriate treatment such as concentration, dilution, drying, dissolution, fractionation, extraction, and purification, as required. That is, as PHS, for example, a product purified to a desired extent may be used, or a material containing PHS may be used. The material containing PHS is not particularly limited so long as the conversion of PHS to PHC proceeds. Specific examples of the material containing PHS include a culture broth containing PHS, a supernatant separated from the culture broth, and processed products thereof such as concentrated products (such as concentrated liquid) thereof and dried products thereof.

Methods for converting PHS to PHC are not particularly limited.

PHS can be converted to PHC by, for example, a chemical reaction with a fatty acid (U.S. Pat. No. 5,869,711). The fatty acid is not particularly limited so long as it provides the acyl chain of the PHC to be produced. That is, examples of the fatty acid include those corresponding to the acyl chains of the PHC species exemplified above. Specific examples of the fatty acid include myristic acid (14:0), palmitic acid (16:0), stearic acid (18:0), arachidic acid (20:0), behenic acid (22:0), lignoceric acid (24:0), cerotic acid (26:0), myristoleic acid (14:1), palmitoleic acid (16:1), oleic acid (18:1), linoleic acid (18:2), and linolenic acid (18:3). Particular examples of the fatty acid include stearic acid (18:0). As PHS, a single kind of PHS species may be used, or two or more kinds of PHS species may be used in combination. As the fatty acid, a single kind of fatty acid may be used, or two or more kinds of fatty acids may be used in combination. Use of two or more kinds of PHS species and/or two or more kinds of fatty acids may result in production of a mixture of two or more kinds of PHC species.

Confirmation of the production of PHC and collection of PHC can be carried out in the same manners as those described herein. That is, this method for producing PHC may further include collecting PHC. The purity of PHC collected may be, for example, 30% (w/w) or higher, 50% (w/w) or higher, 70% (w/w) or higher, 80% (w/w) or higher, 90% (w/w) or higher, or 95% (w/w) or higher.

### EXAMPLES

The present invention will be more specifically explained with reference to the following non-limiting examples.

Materials used in the Examples are shown in Tables 1-6.

TABLE 1
Primers

| Primers | SEQ ID NO |
|---|---|
| EV4215 | 41 |
| EV4216 | 42 |
| EV3782 | 43 |
| EV3783 | 44 |
| AG1009 | 45 |
| AG1010 | 46 |
| AG1011 | 47 |
| AG1021 | 48 |
| AG1022 | 49 |
| AG1023 | 50 |
| AG1091 | 51 |
| AG1092 | 52 |
| AG1013 | 53 |
| AG1014 | 54 |
| AG1015 | 55 |
| NI73 | 56 |
| NI74 | 57 |
| NI86 | 58 |
| NI98 | 59 |
| NI87 | 60 |
| NI99 | 61 |
| EK238 | 62 |
| EK249 | 63 |
| EK250 | 64 |
| EK254 | 65 |

TABLE 2
Promoters

| Promoter | SEQ ID NO |
|---|---|
| GPD1 | 66 |
| TEF2 | 67 |
| PGK1 | 68 |
| TPI1 | 69 |

TABLE 3
Terminators

| Terminator | SEQ ID NO |
|---|---|
| CYC1 | 70 |
| PGI1 | 71 |
| ADH2 | 72 |
| TDH1 | 73 |
| ENO2 | 74 |

TABLE 4
Plasmids

| Plasmid | SEQ ID NO |
|---|---|
| DEVE0078 | 75 |
| pNI-nat | 76 |
| pNI-hph | 77 |
| pUC57-KIURA3-PTDH3-L169R | 78 |
| pUG-PTDH3 | 79 |
| pUC19-RS-HIS3-RS-PADH1 | 80 |
| pUC19-RS-LEU2-RS-PADH1 | 81 |
| pUC18-loxP-nat-loxP | 82 |
| pAC004-ble-pPGK1-Cre | 83 |

TABLE 5
Complete SC mixture

| Component | Concentration (mg/L)* |
|---|---|
| Adenine | 18 |
| L-Alanine | 74 |
| L-Arginine HCl | 74 |
| L-Asparagine | 74 |
| L-Aspartic Acid | 74 |
| L-Cysteine | 74 |
| L-Glutamine | 74 |
| L-Glutamic Acid | 74 |
| Glycine | 74 |
| L-Histidine | 74 |
| myo-Inositol | 74 |
| L-Isoleucine | 74 |
| L-Leucine | 367 |
| L-Lysine | 74 |
| DL-Methionine | 74 |
| para-Aminobenzoic Acid | 74 |
| L-Phenylalanine | 74 |
| L-Proline | 74 |
| L-Serine | 74 |
| L-Threonine | 74 |
| L-Tryptophan | 74 |
| L-Tyrosine | 74 |
| Uracil | 74 |
| L-Valine | 74 |

*Final concentration when the mixture is used in an amount of 2.0 g/L.

TABLE 6
SC mix without leucine, histidine, and uracil

| Component | Concentration (mg/L)* |
|---|---|
| Adenine | 25 |
| L-Alanine | 99 |
| L-Arginine HCl | 99 |
| L-Asparagine | 99 |
| L-Aspartic Acid | 99 |
| L-Cysteine | 99 |
| L-Glutamine | 99 |
| L-Glutamic Acid | 99 |
| Glycine | 99 |
| myo-Inositol | 99 |
| L-Isoleucine | 99 |
| L-Lysine | 99 |
| DL-Methionine | 99 |
| para-Aminobenzoic Acid | 99 |
| L-Phenylalanine | 99 |
| L-Proline | 99 |
| L-Serine | 99 |
| L-Threonine | 99 |
| L-Tryptophan | 99 |
| L-Tyrosine | 99 |
| L-Valine | 99 |

*Final concentration when the mixture is used in an amount of 2.0 g/L.

### Example 1: Construction of Strains

Saccharomyces cerevisiae strain SCP4510, the most developed PHS (Phytosphingosine) producer strain, was constructed from strain EYS5009 disclosed in WO2017/033464. Strain EYS5009 is a strain constructed from strain NCYC 3608 and is deficient in the LCB4 and CKA2 genes. Strain NCYC 3608 (genotype MATalpha gal2 ho::HygMX ura3::KanMX) is a Mat a derivative of S288C (ATCC 26108). Strain SCP4510 contains the following modifications, namely the deletion of leu2Δ0 Δcha1::LoxP Δcka2::LoxP Δlcb4::LoxP Δorm2::LoxP Δnem1::LEU2 CAT5-91Met gal2 ho YNRCΔ9::ScLCB1/ScSUR2 YPRCΔ15::ScLCB2/ScTSC10 PTDH3-LCB1 PTDH3-LCB2 PTDH3-TSC10 PTDH3-SUR2 Ser1::PTEF1-SER3-TENO2-PPGK1-SER2-TADH2-PGPD1-SER1. Strain SCP4510 can be manipulated using standard genetic methods and can be used as a regular diploid or haploid yeast strain. The construction of strain SCP4510 is described below in detail.

S. cerevisiae strain EYS5065 was constructed from strain EYS5009 by deletion of ORM2 gene by a PCR-based gene deletion strategy generating a start-to-stop-codon deletion of the open reading frame. The ORM2 gene was replaced by a deletion construct of the nourseothricin resistance gene NatMX (nat1) flanked by loxP sites, and nucleotide sequences homologous to the native promoter and terminator of the ORM2 gene that was added by PCR using primers EV4215 and EV4216 and plasmid pNI-nat as a template. Transformants were selected on SC-agar plates (6.7 g/L yeast nitrogen base w/o amino acids, 2.0 g/L complete SC mixture, 20 g/L glucose, 20 g/L agar) containing 100 mg/L nourseothricin. Clones were tested by PCR for proper insertion of the deletion construct. The clone having the proper insertion was designated as EYS5065.

S. cerevisiae strain EYS5180 was constructed from the previously described strain EYS5065 by deletion of the CHA1 gene by a PCR-based gene deletion strategy generating a start-to-stop-codon deletion of the open reading frame. The CHA1 gene was replaced by a deletion construct of the hygromycin resistance gene HygMX (Hph) gene flanked by loxP sites, and nucleotide sequences homologous to the native promoter and terminator of the CHA1 gene that were added by PCR using primers EV3782 and EV3783 and plasmid pNI-hph as a template. Transformants were selected on SC-agar plates containing 100 mg/L hygromycin. Clones were verified by PCR testing for proper insertion of the deletion construct. Additionally, the resistance markers NatMX and HygMX previously used to delete the ORM2 and CHA1 genes, respectively, were removed from a clone having the proper insertion by transformation with pEVE0078, which is a URA3 selectable plasmid containing an expression cassette for the Cre recombinase. Cre recombinase catalyzes site specific recombination between two loxP sites flanking the above described markers with concomitant removal of the same. Clones expressing the Cre recombinase were selected on SC-agar plates without uracil. A few clones were picked and tested for the loss of the selection markers by plating on the respective selective plates. The plasmid pEVE0078 was removed by growing strains in the presence of 1 g/L 5′-fluoroorotic acid, which is converted into a toxic compound by the activity of the URA3 gene product. Only clones that had lost the plasmid pEVE0078 were able to grow on the medium containing 5′-fluoroorotic acid. The grown clone was designated as EYS5180.

S. cerevisiae strain EVST20075 was constructed from the previously described strain EYS5180 by introduction of 2 integration modules. The first integration module of native S. cerevisiae LCB1 and SUR2 genes and the selectable marker NatMX was integrated into the genomic Tyl long-terminal repeat YNRCA9 (Chromosome XIV 727363-727661). LCB1 and SUR2 genes were expressed from native S. cerevisiae GPD1 and TEF2 promoters, respectively, followed by native S. cerevisiae CYC1 and PGI1 terminators. In addition, the second integration module of native S. cerevisiae LCB2 and TSC10 genes and the selectable marker HygMX (Hph) was integrated into the genomic Tyl long-terminal repeat YPRCA15 (Chromosome XVI 776667 . . . 776796). LCB2 and TSC10 genes were expressed from native S. cerevisiae PGK1 and TPI1 promoters, respectively, followed by native S. cerevisiae ADH2 and TDH1 terminators. The clone having the integration modules was designated as EVST20075. The nucleotide sequences of the integrated modules were analyzed and 3 nucleotides-lacking was found in the open reading frame of LCB2 (lacking the 1438^{th} to 1440^{th} nucleotide).

S. cerevisiae strain AGRI-536 was constructed from the previously described strain EVST20075 by replacement of the promoter of LCB1 at the original locus with the promoter of TDH3 of S. cerevisiae (PTDH3). For promoter replacement, a cassette of K1URA3 (URA3 gene of Kluyveromyces lactis) and PTDH3 was integrated upstream of LCB1 open reading frame in such a way that the 3′ end of PTDH3 was connected with 5′ end of LCB1 open reading frame. This cassette also contained the 169 bp of PTDH3 5′ end, located upstream of K1URA3 in the same orientation as full-sized PTDH3. To integrate this cassette, a DNA fragment having a LCB1 upstream region was generated by PCR using primers AG1009 and AG1010 and chromosomal DNA of strain S. cerevisiae S288C as a template. This DNA fragment was mixed with plasmid pUC57-KlURA3-PTDH3-L169R, and this mixture was used as a template for PCR with primers AG1009 and AG1011. The product of PCR was used for transformation of strain EVST20075, and transformants were selected on SC-agar plates without uracil. Transformants were tested by PCR for proper insertion of the promoter replacement construct. The clone having the proper insertion was designated as AGRI-536. The nucleotide sequence of PTDH3 promoter integrated upstream of LCB1 was confirmed by sequence analysis.

S. cerevisiae strain AGRI-537 was constructed from the previously described strain AGRI-536 by removing K1URA3 gene previously used to replace LCB1 promoter. Removing of K1URA3 occurred due to homologous recombination between 169 bp PTDH3 5′ region located upstream of K1URA3 and 5′ region of full-sized PTDH3 located downstream of this gene. Selection of strains with loss of K1URA3 was carried out by growing of AG-536 on SC-agar plates supplemented with 1 g/L 5′-fluoroorotic acid. In the presence of this compound, only strains with inactivated URA3 survived. Removing of K1URA3 was confirmed with PCR analysis. The clone from which K1URA3 was removed was designated as AGRI-537.

S. cerevisiae strain AGRI-526 was constructed from the previously described strain AGRI-537 by replacement of the promoter of SUR2 at the original locus by PTDH3. The procedure of promoter replacement was the same as the procedure described above for replacement of the promoter of LCB1 at the original locus, except that primers AG1021 and AG1022 were used for amplification of SUR2 upstream region and primers AG1021 and AG1023 were used for generation of DNA fragment for transformation. The resulting clone was designated as AGRI-526.

S. cerevisiae strain AGRI-528 was constructed from the previously described strain AGRI-526 by removing K1URA3 previously used to replace SUR2 promoter. The procedure of K1URA3 elimination was the same as the procedure described above for elimination of K1URA3 used for replacement of promoter of LCB1. The resulting clone was designated as AGRI-528.

S. cerevisiae strain AGRI-534 was constructed from the previously described strain AGRI-528 by replacement of the promoter of TSC10 at the original locus by PTDH3. For promoter replacement, a cassette of geneticin (G418) resistance gene KanMX flanked by loxP sites and PTDH3 was integrated upstream of TSC10 open reading frame in such a way that the 3′ end of PTDH3 was connected with 5′ end of TSC10 open reading frame. To integrate this cassette, a DNA fragment was synthesized by PCR using primers AG1091 and AG1092 and pUG-PTDH3 as a template. Resulting DNA fragment was used for transformation of AGRI-528, and transformants were selected on YPD-agar plates (10 g/l yeast extract, 20 g/L bacto-peptone, 20 g/L glucose, 20 g/L agar) supplemented with 200 mg/L of G418. Transformants were tested by PCR for proper insertion of the promoter replacement construct. The clone having the proper insertion was designated as AGRI-534. The nucleotide sequence of PTDH3 integrated upstream of TSC10 was confirmed by sequence analysis.

S. cerevisiae strain AGRI-551 was constructed from the previously described strain AGRI-534 by replacement of the promoter of LCB2 at the original locus by PTDH3. The procedure of promoter replacement was the same as the procedure described above for replacement of the promoter of LCB1 at the original locus, except that primers AG1013 and AG1014 were used for amplification of LCB2 upstream region and primers AG1013 and AG1015 were used for generation of DNA fragment for transformation. The resulting clone was designated as AGRI-551.

S. cerevisiae strain SCP1100 was constructed from the previously described strain AGRI-551 by introduction of S. cerevisiae HIS3 gene into the upstream of the ERG3 open reading frame. The HIS3 gene was introduced with nucleotide sequences homologous to the upstream of the ERG3 open reading frame that were added by PCR using primers N173 and N174 with the plasmid pUC19-RS-HIS3-RS-PADH1 as a template. Transformants were selected on SC-agar plates without histidine. Clones were tested by PCR for proper insertion of HIS3. The clone having the proper insertion was designated as SCP1100.

S. cerevisiae strain SCP1300 was constructed from the previously described strain SCP1100 by introduction of the S. cerevisiae LEU2 gene into the upstream of the NEM1 open reading frame. The LEU2 gene was introduced with nucleotide sequences homologous to the upstream of the NEM1 open reading frame that were added by PCR using primers N186 and NI98 with the plasmid pUC19-RS-LEU2-RS-PADH1 as PCR template. Transformants were selected on SC-agar plates without leucine. Clones were tested by PCR for proper insertion of LEU2. The clone having the proper insertion was designated as SCP1300.

S. cerevisiae strain SCP1400 was constructed from the previously described strain SCP1100 by deletion of the NEM1 gene by a PCR-based gene deletion strategy. The NEM1 gene was replaced with a deletion construct of S. cerevisiae LEU2 gene and nucleotide sequences homologous to the NEM1 open reading frame that were added by PCR using primers NI87 and NI99 with the plasmid pUC19-RS-LEU2-RS-PADH1 as a template. Transformants were selected on SC-agar plates without leucine. Clones were tested by PCR for proper insertion of the deletion construct. The clone having the proper insertion was designated as SCP1400.

S. cerevisiae strain SCP2400 was constructed from the previously described strain SCP1400 by removing K1URA3 gene previously used to replace LCB2 promoter. The K1URA3 gene was removed by growing strains in the presence of 1 g/L 5′-fluoroorotic acid. Only clones that had lost the K1URA3 gene were able to grow on the medium containing 5′-fluoroorotic acid. These grown clones were designated as SCP2400. Additionally, the resistance markers NatMX and HygMX previously used to integrate the expressing module of LCB1/SUR2 into YNRCA9 and of LCB2/ScTSC10 into YPRCA15, respectively, were removed from SCP2400 by transformation with pEVE0078. A few clones were picked and tested for the loss of the selection markers by plating on the respective selective plates. The plasmid pEVE0078 was removed by growing strains in the presence of 1 g/L 5′-fluoroorotic acid. The clone which was able to grow on the medium containing 5′-fluoroorotic acid was selected and designated as SCP2410.

S. cerevisiae strain SCP3410 was constructed from the previously described strain SCP2410 by restoring the S. cerevisiae URA3 gene (ScURA3) into the original Ura3 locus. The ScURA3 gene was introduced with nucleotide sequences homologous to the upstream and the downstream of the ScURA3 open reading frame. The DNA fragment for transformation was prepared by PCR using primers EK238 and EK249 with chromosomal DNA of strain S. cerevisiae S288C as a template. Transformants were selected on SC-agar plates without uracil. Clones were tested by PCR for proper insertion of ScURA3. The clone having the proper insertion was designated as SCP3410.

S. cerevisiae strain SCP3900 was constructed from the previously described strain SCP3410 by replacing the promoter of YPC1 gene by PTDH3. The YPC1 promoter was replaced by a promoter replacing construct of the NatMX flanked by loxP sites, and nucleotide sequences homologous to the native promoter of the YPC1 gene that was added by PCR using primers EK250 and EK254 with the plasmid pUC18-loxP-nat-loxP as a template. Transformants were selected on SC-agar plates containing 100 mg/L nourseothricin. Clones were tested by PCR for proper insertion of the promoter replacement construct. The clone having the proper insertion was designated as SCP3900.

S. cerevisiae strain SCP4100 was constructed from the previously described strain SCP3410 by introduction of an integration module of S. cerevisiae SER3, SER2, and SER1 genes and the selectable marker KanMX. The integration module was integrated into the original SER1 locus. SER3, SER2, and SER1 genes were expressed from native S. cerevisiae TEF1, ENO2, and GPD1 promoters, respectively, followed by native S. cerevisiae ENO2 and ADH2 terminators. The clone having the integration module was designated as SCP4100.

S. cerevisiae strain SCP4500 was constructed from the previously described strain SCP4100 by replacing the 3 nucleotides-lacking LCB2 expression module in YPRCA15 locus with another LCB2 expression module with a correct LCB2 nucleotide sequence. The already integrated 3 nucleotides-lacking LCB2 expression module was replaced with an integration module of S. cerevisiae LCB2 gene and the selectable marker HygMX. LCB2 gene was expressed from native S. cerevisiae PGK1 promoter, followed by native S. cerevisiae ADH2 terminator. Additionally, the resistance markers KanMX and HygMX previously used were removed from a clone having proper insertion of the integration module by transformation with pAC004-ble-pPGK1-Cre, which is a plasmid with zeocin (a copper-chelated glycopeptide antibiotic, invitrogen) selectable antibiotic marker containing an expression cassette for the Cre recombinase. A few clones were picked and tested for the loss of the selection markers by plating on the respective selective plates. The plasmid pAC004-ble-pPGK1-Cre was removed by growing strains in the SC-agar plate without antibiotics. The clone which was not able to grow on the medium containing zeocin was selected and designated as SCP4510.

### Example 2: Cultivation of Strains in Small Scale Batch Culture and Analysis for PHS Production

Constructed strains were each streaked as patches on selective SC-agar plates lacking leucine, histidine, and uracil and containing 15 g/L of alpha-cyclodextrin. After 2 overnight growth periods, the strains were each inoculated into flasks with 50 mL of SCA-medium lacking leucine, histidine, and uracil (1.45 g/L SC-mix without leucine, histidine and uracil, 1.7 g/L yeast nitrogen base, 5 g/l ammonium sulfate, 20 g/L glucose, 15 g/L alpha-cyclodextrin, pH 5.2). After 30 hours of incubation at 30° C. with shaking, the culture broths were harvested. The culture broths were diluted in 15 g/L alpha-cyclodextrin aqueous solution so that the concentrations of analytes were within the calibration range.

A series of calibration solutions at 1 mg/L, 0.5 mg/L, 0.25 mg/L, 0.125 mg/L, 62.5 μg/L, and 31.25 μg/L of phytosphingosine (Tokyo Chemical Industry Co., Ltd., Product Number P1765, 5 g) in 15 g/L alpha-cyclodextrin aqueous solution was prepared and injected into the LC-MS/MS. The LC-MS/MS conditions were as follows: Mobile Phase A, 2 mM ammonium formate in water+0.2% formic acid; Mobile Phase B, 1 mM ammonium formate in acetonitrile/methanol 1:1+0.2% formic acid; Column, Acquity BEH UPLC C8, 2.1×100 mm, 1.7 m. The flow rate was set at 0.4 mL/min. The elution gradient is shown in Table 1. Phytosphingosine was detected as product ion m/z 60.1 from precursor ion m/z 318.3 by Electrospray Positive Mode (ESI+). The concentration of phytosphingosine was calculated according to the calibration curve.

TABLE 7
Elution Gradient

| Time | % B |
|---|---|
| 0.0 | 50 |
| 1.0 | 85 |
| 4.0 | 100 |
| 4.7 | 100 |
| 4.8 | 50 |
| 5.5 | 50 |

Phytosphingosine production for strains SCP1300, SCP1400, SCP3410, SCP3900, SCP4100, SCP4500, and SCP4510 were measured. Results are shown in Table 8. The strain with nem1 deletion (SCP1400) showed higher phytosphingosine production than that of the control strain (SCP1300). In addition, the strain introduced with SER3, SER2, and SER1 genes (SCP4100) showed further improvement in phytosphingosine production as compared to the strain without such a genetic modification (SCP3410).

TABLE 8
Phytosphingosine titers in small scale cultures

| Strain | Phytosphingosine (mg/L) |
|---|---|
| SCP1300 | 48.3 ± 2.3 |
| SCP1400 | 62.4 ± 2.3 |
| SCP3410 | 63.8 ± 3.9 |
| SCP3900 | 66.8 ± 1.1 |
| SCP4100 | 73.5 ± 3.2 |
| SCP4500 | 73.7 ± 1.4 |
| SCP4510 | 75.9 ± 1.1 |

Average of 3 flasks, ± shows s.d.

### INDUSTRIAL APPLICABILITY

According to the present invention, an ability of yeast to produce an objective substance such as phytosphingosine (PHS) and phytoceramide (PHC) can be improved, and an objective substance can be efficiently produced.

### <Explanation of Sequence Listing>

SEQ ID NO: 1, Nucleotide sequence of LCB1 gene of Saccharomyces cerevisiae
SEQ ID NO: 2, Amino acid sequence of Lcb1 protein of Saccharomyces cerevisiae
SEQ ID NO: 3, Nucleotide sequence of LCB2 gene of Saccharomyces cerevisiae
SEQ ID NO: 4, Amino acid sequence of Lcb2 protein of Saccharomyces cerevisiae
SEQ ID NO: 5, Nucleotide sequence of TSC10 gene of Saccharomyces cerevisiae
SEQ ID NO: 6, Amino acid sequence of Tsc10 protein of Saccharomyces cerevisiae
SEQ ID NO: 7, Nucleotide sequence of SUR2 gene of Saccharomyces cerevisiae
SEQ ID NO: 8, Amino acid sequence of Sur2 protein of Saccharomyces cerevisiae
SEQ ID NO: 9, Nucleotide sequence of SUR2 gene of Pichia ciferrii
SEQ ID NO: 10, Amino acid sequence of Sur2 protein of Pichia ciferrii
SEQ ID NO: 11, Nucleotide sequence of LAG1 gene of Saccharomyces cerevisiae
SEQ ID NO: 12, Amino acid sequence of Lag1 protein of Saccharomyces cerevisiae
SEQ ID NO: 13, Nucleotide sequence of LAC1 gene of Saccharomyces cerevisiae
SEQ ID NO: 14, Amino acid sequence of Lac protein of Saccharomyces cerevisiae
SEQ ID NO: 15, Nucleotide sequence of LIP1 gene of Saccharomyces cerevisiae
SEQ ID NO: 16, Amino acid sequence of Lip1 protein of Saccharomyces cerevisiae
SEQ ID NO: 17, Nucleotide sequence of SER1 gene of Saccharomyces cerevisiae
SEQ ID NO: 18, Amino acid sequence of Ser1 protein of Saccharomyces cerevisiae
SEQ ID NO: 19, Nucleotide sequence of SER2 gene of Saccharomyces cerevisiae
SEQ ID NO: 20, Amino acid sequence of Ser2 protein of Saccharomyces cerevisiae
SEQ ID NO: 21, Nucleotide sequence of SER3 gene of Saccharomyces cerevisiae
SEQ ID NO: 22, Amino acid sequence of Ser3 protein of Saccharomyces cerevisiae
SEQ ID NO: 23, Nucleotide sequence of YPC1 gene of Saccharomyces cerevisiae
SEQ ID NO: 24, Amino acid sequence of Ypc1 protein of Saccharomyces cerevisiae
SEQ ID NO: 25, Nucleotide sequence of NEM1 gene of Saccharomyces cerevisiae
SEQ ID NO: 26, Amino acid sequence of Nem1 protein of Saccharomyces cerevisiae
SEQ ID NO: 27, Nucleotide sequence of SPO7 gene of Saccharomyces cerevisiae
SEQ ID NO: 28, Amino acid sequence of Spo7 protein of Saccharomyces cerevisiae
SEQ ID NO: 29, Nucleotide sequence of LCB4 gene of Saccharomyces cerevisiae
SEQ ID NO: 30, Amino acid sequence of Lcb4 protein of Saccharomyces cerevisiae
SEQ ID NO: 31, Nucleotide sequence of LCB5 gene of Saccharomyces cerevisiae
SEQ ID NO: 32, Amino acid sequence of Lcb5 protein of Saccharomyces cerevisiae
SEQ ID NO: 33, Nucleotide sequence of ELO3 gene of Saccharomyces cerevisiae
SEQ ID NO: 34, Amino acid sequence of Elo3 protein of Saccharomyces cerevisiae
SEQ ID NO: 35, Nucleotide sequence of CKA2 gene of Saccharomyces cerevisiae
SEQ ID NO: 36, Amino acid sequence of Cka2 protein of Saccharomyces cerevisiae
SEQ ID NO: 37, Nucleotide sequence of ORM2 gene of Saccharomyces cerevisiae
SEQ ID NO: 38, Amino acid sequence of Orm2 protein of Saccharomyces cerevisiae
SEQ ID NO: 39, Nucleotide sequence of CHA1 gene of Saccharomyces cerevisiae
SEQ ID NO: 40, Amino acid sequence of Cha1 protein of Saccharomyces cerevisiae
SEQ ID NOS: 41-65, Primers
SEQ ID NOS: 65-69, Promoters
SEQ ID NOS: 70-74, Terminators
SEQ ID NOS: 75-83, Plasmids

1. A method for producing an objective substance, the method comprising:
cultivating yeast having an ability to produce the objective substance in a culture medium,
wherein the yeast has been modified so that expression and/or activity of a protein(s) encoded by NEM1 and/or SPO7 gene(s) is reduced as compared with a non-modified strain, and
wherein the objective substance is selected from the group consisting of phytosphingosine (PHS) and phytoceramide (PHC).

2. The method according to claim 1, wherein at least the expression and/or activity of the protein encoded by the NEM1 gene is reduced.

3. The method according to claim 1, wherein the activity of each of the protein(s) is reduced by reducing the expression of the gene encoding the protein, or by disrupting the gene encoding the protein.

4. The method according to claim 1, wherein the expression and/or activity of each of the protein(s) is reduced by deletion of the gene encoding the protein.

5. The method according to claim 1, wherein the protein encoded by the NEM1 gene is selected from the group consisting of:
(A) a protein comprising the amino acid sequence of SEQ ID NO: 26;
(B) a protein comprising the amino acid sequence of SEQ ID NO: 26 but including substitution, deletion, insertion, and/or addition of 1 to 10 amino acid residues, and functioning as a catalytic subunit of Nem1-Spo7 protein phosphatase;
(C) a protein comprising an amino acid sequence having 90% or higher sequence identity to the amino acid sequence of SEQ ID NO: 26, and functioning as a catalytic subunit of Nem1-Spo7 protein phosphatase.

6. The method according to claim 1, wherein the protein encoded by the SPO7 gene is selected from the group consisting of:
(A) a protein comprising the amino acid sequence of SEQ ID NO: 28;
(B) a protein comprising the amino acid sequence of SEQ ID NO: 28 but including substitution, deletion, insertion, and/or addition of 1 to 10 amino acid residues, and functioning as a regulatory subunit of Nem1-Spo7 protein phosphatase; (C) a protein comprising an amino acid sequence having 90% or higher sequence identity to the amino acid sequence of SEQ ID NO: 28, and functioning as a regulatory subunit of Nem1-Spo7 protein phosphatase.

7. The method according to claim 1,
wherein the objective substance is PHS, and the yeast has further been modified so that expression and/or activity of one or more proteins encoded by a gene selected from the group consisting of LAG1, LAC1, LIP1, LCB4, LCB5, ELO3, CKA2, ORM2, CHA1, and combinations thereof is reduced as compared with a non-modified yeast, or
wherein the objective substance is PHC, and the yeast has further been modified so that expression and/or activity of one or more proteins encoded by a gene selected from the group consisting of YPC1, LCB4, LCB5, ORM2, CHA1, and combinations thereof is reduced as compared with a non-modified yeast.

8. The method according to claim 7, wherein the activity of each of the one or more proteins is reduced by reducing the expression of the gene encoding the protein, or by disrupting the gene encoding the protein.

9. The method according to claim 7, wherein expression and/or activity of each of the one or more proteins is reduced by deletion of the gene encoding the protein.

10. The method according to claim 1,
wherein the objective substance is PHS, and the yeast has further been modified so that expression and/or activity of one or more proteins encoded by a gene selected from the group consisting of LCB1, LCB2, TSC10, SUR2, SER1, SER2, SER3, YPC1, and combinations thereof is increased as compared with a non-modified yeast, or
wherein the objective substance is PHC, and the yeast has further been modified so that expression and/or activity of one or more proteins encoded by a gene selected from the group consisting of LCB1, LCB2, TSC10, SUR2, LAG1, LAC1, LIP1, SER1, SER2, SER3, ELO3, and combination thereof is increased as compared with a non-modified strain.

11. The method according to claim 10, wherein at least the expression and/or activity of the protein encoded by the YPC1 gene is increased.

12. The method according to claim 10, wherein at least the expression and/or activity of one or more proteins encoded by a gene selected from the group consisting of SER1, SER2, SER3, and combinations thereof is increased.

13. The method according to claim 10, wherein the activity of each of the one or more proteins is increased by increasing the expression of the gene encoding the protein.

14. The method according to claim 10, wherein the expression and/or activity of each of the one or more proteins is increased by increasing the copy number of the gene encoding the protein, and/or by modifying an expression control sequence of the gene encoding the protein.

15. The method according to claim 1, wherein the PHS is selected from the group consisting of C16:0 PHS, C18:0 PHS, C20:0 PHS, C18:1 PHS, C20:1 PHS, 4-(hydroxymethyl)-2-methyl-6-tetradecanyl-1,3-oxazinan-5-ol, and 4-(hydroxymethyl)-2-methyl-6-hexadecanyl-1,3-oxazinan-5-ol.

16. The method according to claim 15, wherein the culture medium contains an additive that is able to associate with, bind to, solubilize, and/or capture the objective substance.

17. The method according to claim 16, wherein the additive is selected from the group consisting of cyclodextrin and zeolite.

18. The method according to claim 1, wherein the yeast belongs to the genus Saccharomyces.

19. The method according to claim 1, wherein the yeast is Saccharomyces cerevisiae.

20. The method according to claim 1, wherein the yeast is able to produce and accumulate the objective substance in a culture medium or cells of the yeast in an amount larger than that obtainable with a non-modified yeast.

21. The method according to claim 1, the method further comprising:
collecting the objective substance from cells of the yeast and/or the culture medium.

22. A method for producing phytoceramide (PHC), the method comprising:
producing phytosphingosine (PHS) by the method according to claim 1; and
converting the PHS to the PHC.
